(19) United States
(12) Patent Application Publication
(10) Pub. No.: US 2026/0258010 A1
(43) Pub. Date: Sep. 3, 2026

(54) THIOPHENE-2-CARBOXYLIC ACID DERIVATIVE, AND PREPARATION METHOD THEREFOR AND MEDICAL USE THEREOF

(71) Applicant: CHINA PHARMACEUTICAL UNIVERSITY, Jiangsu (CN)

(72) Inventors: Cheng JIANG, Jiangsu (CN); Qinghua HU, Jiangsu (CN); Yuhang WANG, Jiangsu (CN); Mengze ZHOU, Jiangsu (CN); Pingping WANG, Jiangsu (CN); Rui GAO, Jiangsu (CN); Xinyue LI, Jiangsu (CN)

(73) Assignee: CHINA PHARMACEUTICAL UNIVERSITY, Jiangsu (CN)

(21) Appl. No.: 19/161,306

(22) PCT Filed: Jul. 24, 2024
(86) PCT No.: PCT/CN2024/107199

§ 371 Date: Sep. 1, 2025

(30) Foreign Application Priority Data
Jun. 14, 2023 (CN) 202310699720.0

(51) Int. Cl. C07D 333/40; A61K 31/381; A61K 31/4436; A61K 31/506; A61P 29/00; C07D 409/12; C07D 409/14; C07D 417/12

(52) U.S. Cl. CPC C07D 333/40; A61K 31/381; A61K 31/4436; A61K 31/506; A61P 29/00; C07D 409/12; C07D 409/14; C07D 417/12

(57) ABSTRACT

Provided are a thiophene-2-carboxylic acid derivative, and a preparation method therefor and the medical use thereof. Also provided are a thiophene-2-carboxylic acid derivative as represented by general formula (I), and an enantiomer, diastereoisomer, tautomer, N-oxide, solvate, physiologically hydrolysable ester, preparation and pharmaceutically acceptable salt thereof. Further provided are a thiophene-2-carboxylic acid derivative having an inhibitory effect on a P2Y14 receptor and a pharmaceutically acceptable salt thereof. Pharmacological experiments show that the compound has a significant inhibitory effect on the P2Y14 receptor, and can be particularly used as a drug for treating inflammatory diseases.

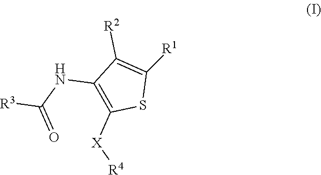

### TECHNICAL FIELD

The present disclosure relates to a compound, a preparation method therefor, and use thereof, and in particular to a thiophene-2-carboxylic acid derivative, a preparation method therefor, and pharmaceutical use thereof.

### BACKGROUND OF RELATED ART

When the body is stressed or damaged, various important intracellular molecules, such as adenosine-5′-triphosphate (ATP) and uridine-5′-triphosphate (UTP), are released into the extracellular fluid by specific tissues or organs. These nucleotides have the ability to regulate innate and adaptive immune responses by binding to cell surface receptors. The bound receptors are identified as purinergic receptors, which are mainly divided into adenosine (P1) and nucleotide (P2) receptors. The P2 family is further divided into two subfamilies, the P2X receptors and the P2Y receptors (P2YRs). P2YRs belong to the family of G protein-coupled receptors (GPCRs), consisting of P2Y1-like receptors (P2Y_{1, 2, 4, 6, and 11}) and P2Y12-like receptors (P2Y_{12, 13, and 14}). The reported P2Y receptor family of G protein-coupled receptors comprises 8 subtypes (P2Y_{1, 2, 4, 6, 11, 12, 13, and 14}), which are widely distributed in various cells and tissues. Moreover, the homology among the subtypes is relatively low, and thus, different subtypes have high selectivity for ligands. Among them, the P2Y_{1, 2, 4, and 6} receptors bind to G_{q} and activate the PLC pathway; the P2Y_{12, 13, and 14} receptors bind to Gᵢ to inhibit the activity of adenylate cyclase; the P2Y₄ receptor is coupled to two G proteins G_{q}/Gᵢ; and the P2Y₁₁ is coupled to two G proteins G_{q}/Gₛ. The P2Y receptors mediate a series of biological effects such as immunoregulation, platelet aggregation, and smooth muscle cell proliferation.

The P2Y₁₄ receptor is activated by at least four naturally occurring UDP-sugars, specifically UDP-glucose (UDPG). The P2Y₁₄ receptor has been found to be widely expressed in a range of human tissues, including brain, heart, adipose tissue, placenta, intestinal tract, and hematopoietic stem cells. The P2Y₁₄ receptor inhibits adenylate cyclase (AC) by activating the Gᵢ protein to which it is coupled, thereby reducing the production of 3′, 5′-cyclic adenosine monophosphate (cAMP) in cells and the corresponding biological effects.

The P2Y₁₄ receptor has been proven to be a potential target of innate immune inflammatory diseases, such as diabetes, cystic fibrosis, renal aseptic inflammation, acute gouty arthritis, and allergic diseases. The P2Y₁₄ receptor can promote the recruitment and chemotaxis of neutrophils and macrophages, releasing pro-inflammatory cytokines, chemokines, and mast cell mediators. In severe cases caused by COVID-19, a large proportion of the severe course is caused by systemic inflammation, which is known as a “cytokine storm”. Based on the effects of the P2Y₁₄ receptor on neutrophils and the high levels of UDPG found in patients with severe COVID-19, a mechanism to prevent uncontrolled chemotaxis of neutrophils through P2Y₁₄ receptor antagonists has been proposed to avoid cytokine storms and systemic inflammation, thereby reducing patient mortality. In addition, knockout of the P2Y₁₄ receptor gene can inhibit the recruitment of macrophages and tissue inflammation, and alleviate insulin resistance induced by high-fat diets. Another study has shown that glycogen metabolism also increases the level of UDPG in macrophages and the number of P2Y₁₄ receptors, and blocking this glycogen metabolic pathway can effectively inhibit lipopolysaccharide (LPS)-induced acute peritonitis. In summary, the P2Y₁₄ receptor may be a potential therapeutic target for diseases of the innate immune system.

Currently, research on P2Y₁₄ receptor antagonists has reported only 4 structural classes of compounds (pyrimidopiperidines, 2-naphthoic acids, 3-substituted benzoic acids, and pyrazole-3-carboxylic acids), but they are all still in the preclinical research stage. Among them, 2-naphthoic acids have the highest activity and selectivity. However, currently reported antagonists with 2-naphthoic acid structures have the defects of poor solubility, low oral bioavailability, high difficulty in synthesis and purification, and the like, which bring great difficulties to further discussion on the structure-activity relationship and biological evaluation. Therefore, the search for novel structural classes of P2Y₁₄ receptor antagonists to address the problems of poor druggability and the like of 2-naphthoic acid antagonists has emerged as a new strategy for discovering P2Y₁₄ receptor antagonists with strong activity and good selectivity.

### SUMMARY OF THE INVENTION

Objectives: The objective of the present disclosure is to provide a thiophene-2-carboxylic acid derivative with a novel structure and P2Y₁₄ receptor antagonism and a pharmaceutically acceptable salt thereof. Another objective of the present disclosure is to provide a preparation method for the thiophene-2-carboxylic acid derivative described above. Yet another objective of the present disclosure is to provide use of the thiophene-2-carboxylic acid derivative described above in the treatment of an inflammatory disease.

Technical solution: Provided in the present disclosure are a thiophene-2-carboxylic acid derivative of general formula (I), and an enantiomer thereof, a diastereoisomer thereof, a tautomer thereof, an N-oxide thereof, a solvate thereof, a physiologically hydrolyzable ester thereof, a formulation thereof, and a pharmaceutically acceptable salt thereof:

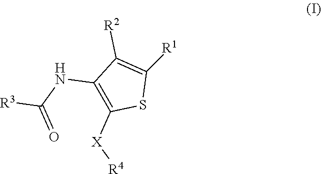

R¹ is selected from CₙH₂ₙ—COOR⁵, CₙH₂ₙ—CONHR⁵, CₙH₂ₙ—CN, and tetrazole, wherein n is selected from 0, 1, and 2;
R² is selected from H and halogen;
R³ is selected from C₅₋₁₀ monocyclic aryl, C₅₋₁₄ monocyclic heteroaryl containing 1-4 heteroatoms selected from N, O, and S, C₃₋₁₀ cycloalkyl, and C₃₋₁₀ heterocycloalkyl, wherein the C₅₋₁₀ monocyclic aryl, C₅₋₁₄ monocyclic heteroaryl containing 1-4 heteroatoms selected from N, O, and S, C₃₋₁₀ cycloalkyl, or C₃₋₁₀ heterocycloalkyl is independently optionally substituted with 1 to 5 substituents selected from: C₁₋₆ alkyl, halogen, cyano, C₁₋₄ haloalkyl, OH, C₁₋₆ alkoxy, and dimethylamino;
X is selected from O, S, and NH;
R⁴ is selected from:

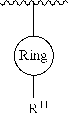

wherein Ring is C₅₋₇ monocyclic aryl or C₅₋₇ monocyclic heteroaryl containing 1-4 heteroatoms selected from N, O, and S;
R⁵ is H, C₂₋₆ alkyl, C₀₋₃OOCCH₃, C₀₋₃OCNMe₂, or C₀₋₃NMe₂, wherein the C₂₋₆ alkyl is unsubstituted or substituted with 1 to 6 substituents selected from: F, OH, C₁₋₃ alkoxy, C₃₋₆ cycloalkyl, and aryl;
wherein R¹¹ is selected from the following substituents: H, NO₂, CN, OH, NH₂, F, Cl, Br, I, C₁₋₆ alkyl, C₁₋₆ haloalkyl, C₁₋₆ alkoxy, C₁₋₆ haloalkoxy, C₀₋₆ heteroalkyl-NR⁷R⁸, C₀₋₆ heteroalkyl-COR⁸, C₀₋₆ heteroalkyl-CO₂R⁸, C₀₋₆ heteroalkyl-CONR⁷R⁸, C₀₋₆ heteroalkyl-SO₃R⁸, and C₀₋₆ heteroalkyl-SO₂NR⁷R⁸; or in R¹¹, two adjacent substituents form a 3- to 8-membered saturated or unsaturated ring containing carbon atoms and optionally containing 1-3 heteroatoms selected from N, O, and S;
R⁷ is H;
R⁸ is selected from H, C₁₋₁₀ alkyl, C₃₋₁₀ cycloalkyl, C₃₋₁₀ heterocycloalkyl, C₁₋₁₀ heteroalkyl-C₃₋₁₀ cycloalkyl, C₁₋₁₀ heteroalkyl-C₃₋₁₀ heterocycloalkyl, and C₁₋₁₀ heteroalkyl-(5-membered heteroaryl), wherein the alkyl, alkylene, cycloalkyl, heterocycloalkyl, and heteroaryl of the C₁₋₁₀ alkyl, C₃₋₁₀ cycloalkyl, C₃₋₁₀ heterocycloalkyl, C₁₋₁₀ heteroalkyl-C₃₋₁₀ cycloalkyl, C₁₋₁₀ heteroalkyl-C₃₋₁₀ heterocycloalkyl, and C₁₋₁₀ heteroalkyl-(5-membered heteroaryl) are unsubstituted or independently substituted with 1-7 substituents selected from: OR⁹, C₁₋₆ alkyl, CO₂R⁹, CONR⁹R¹⁰, NR⁹COR⁹, and C₃₋₁₀ heterocycloalkyl; R⁹ is selected from H and C₁₋₆ alkyl, wherein the C₁₋₆ alkyl is unsubstituted or substituted with 1-6 substituents selected from: F and O—C₁₋₃ alkyl.

Provided is the thiophene-2-carboxylic acid derivative, and the enantiomer thereof, the diastereoisomer thereof, the tautomer thereof, the N-oxide thereof, the solvate thereof, the physiologically hydrolyzable ester thereof, the formulation thereof, and the pharmaceutically acceptable salt thereof, wherein R¹ is selected from CH₂COOR⁵, COOR⁵, and CONHR⁵;
R² is selected from H, F, Cl, and Br;
R³ is selected from C₅₋₆ monocyclic aryl, C₅₋₆ monocyclic heteroaryl containing 1-4 heteroatoms selected from N, O, and S, C₅₋₆ cycloalkyl, and C₅₋₆ heterocycloalkyl, wherein the C₅₋₆ monocyclic aryl, C₅₋₆ monocyclic heteroaryl containing 1-4 heteroatoms selected from N, O, and S, C₅₋₆ cycloalkyl, or C₅₋₆ heterocycloalkyl is independently optionally substituted with 1 to 5 substituents selected from: C₁₋₆ alkyl, halogen, cyano, C₁₋₄ haloalkyl, OH, C₁₋₆ alkoxy, and dimethylamino.

Provided is the thiophene-2-carboxylic acid derivative, and the enantiomer thereof, the diastereoisomer thereof, the tautomer thereof, the N-oxide thereof, the solvate thereof, the physiologically hydrolyzable ester thereof, the formulation thereof, and the pharmaceutically acceptable salt thereof, wherein R¹ is selected from COOR⁵;
R² is selected from H and F;
R³ is selected from phenyl, pyridinyl, pyrazinyl, pyrimidinyl, and imidazolyl, wherein the phenyl, pyridinyl, pyrazinyl, pyrimidinyl, or imidazolyl is independently optionally substituted with 1 to 5 substituents selected from: C₁₋₆ alkyl, halogen, cyano, C₁₋₄ haloalkyl, OH, C₁₋₆ alkoxy, and dimethylamino; X is selected from O, S, and NH;
R⁴ is selected from:

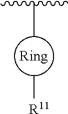

wherein Ring is phenyl, pyridinyl, pyridazinyl, pyrimidinyl, pyrazinyl, pyrrolyl, thienyl, furanyl, thiazolyl, isothiazolyl, imidazolyl, or pyrazolyl;
R⁵ is H, C₂H₅, C₂₋₃ alkyl-C₃₋₆ cycloalkyl, C₂₋₃ alkyl-aryl, C₂₋₃OOCCH₃, C₂₋₃OCNMe₂, or C₂₋₃NMe₂;
R⁷ is H;
R⁸ is selected from H, C₁₋₆ alkyl, C₃₋₆ cycloalkyl, CH₂CONH₂, CH₂COMe₂, CH₂CH₂OH, CH₂CH₂Me, (CH₂)₃OH, (CH₃)₃OMe, (CH₂)₄OH, (CH₂)₄OMe, (CH₂)₅OH, (CH₂)₂COOH, (CH₂)₃COOH, (CH₂)₄COOH, (CH₂)₅COOH, (CH₂)₂CH(CH₃)COOH, C(CH₂OH)₃, C(CH₂OH)₂CH₃, CH₂CH(CH₃)OH, (CH₂)₂CF₃, (CH₂)₃CF₃, (CH₂)₄CF₃,

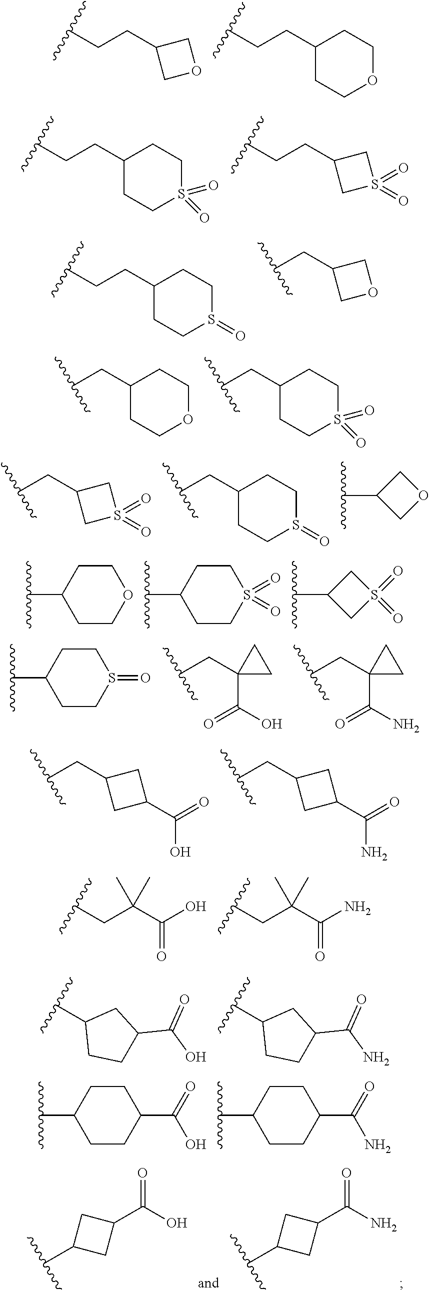

R¹¹ is selected from H, NO₂, CN, OH, NH₂, F, Cl, Br, I, C₁₋₆ alkyl, C₁₋₆ haloalkyl, C₁₋₆ alkoxy, C₁₋₆ haloalkoxy, COR⁸, CONR⁷R⁸, CO₂R⁸, SO₂NR⁷R⁸, and SO₃R⁸.

Provided is the thiophene-2-carboxylic acid derivative, and the enantiomer thereof, the diastereoisomer thereof, the tautomer thereof, the N-oxide thereof, the solvate thereof, the physiologically hydrolyzable ester thereof, the formulation thereof, and the pharmaceutically acceptable salt thereof, wherein R⁴ is selected from:

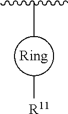

wherein Ring is phenyl, pyrimidinyl, pyridazinyl, or isothiazolyl;
R¹¹ is selected from H, NO₂, CN, OH, NH₂, F, Cl, Br, I, C₁₋₆ alkyl, C₁₋₆ haloalkyl, C₁₋₆ alkoxy, C₁₋₆ haloalkoxy, CONR⁷R⁸, CO₂R⁸, SO₂NR⁷R⁸, and SO₃R⁸;
R⁷ is H;
R⁸ is selected from H, C₁₋₆ alkyl, C₃₋₆ cycloalkyl, CH₂CONH₂, CH₂COMe₂, CH₂CH₂OH, CH₂CH₂Me, (CH₂)₃OH, (CH₃)₃OMe, (CH₂)₄OH, (CH₂)₄OMe, (CH₂)₂COOH, (CH₂)₃COOH, (CH₂)₄COOH, (CH₂)₂CH(CH₃)COOH, C(CH₂OH)₃, C(CH₂OH)₂CH₃, CH₂CH(CH₃)OH,

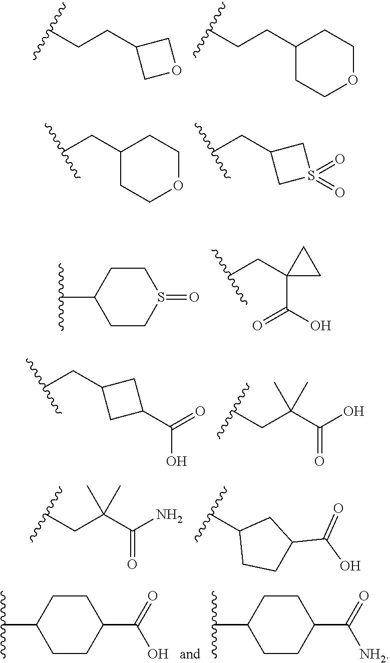

Provided is the thiophene-2-carboxylic acid derivative, and the enantiomer thereof, the diastereoisomer thereof, the tautomer thereof, the N-oxide thereof, the solvate thereof, the physiologically hydrolyzable ester thereof, the formulation thereof, and the pharmaceutically acceptable salt thereof, wherein the compound of general formula (I) is preferably selected from the following compounds:

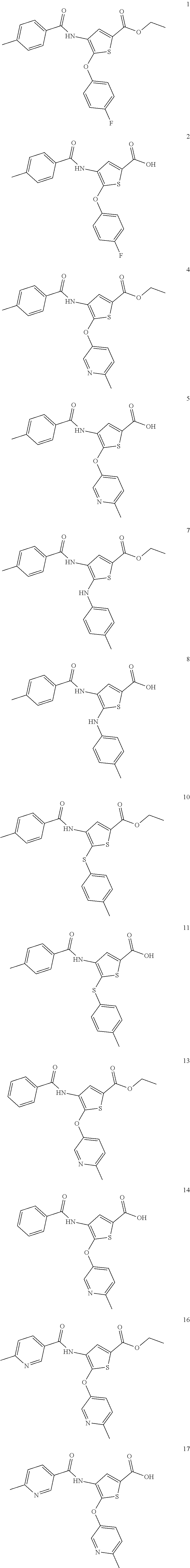

Provided is a preparation method for the thiophene-2-carboxylic acid derivative, and the enantiomer thereof, the diastereoisomer thereof, the tautomer thereof, the N-oxide thereof, the solvate thereof, the physiologically hydrolyzable ester thereof, the formulation thereof, and the pharmaceutically acceptable salt thereof, which comprises the following steps:
(1) subjecting a compound of general formula A to a nitration reaction to prepare a compound of general formula B;
(2) subjecting the compound of general formula B to a substitution reaction with different phenolic derivatives to prepare a compound of general formula C;
(3) subjecting the compound of general formula C to nitro reduction, then to a condensation reaction with different carboxylic acid or sulfonic acid derivatives or to a substitution reaction with different hydrocarbyl derivatives, and finally to a deprotection reaction to prepare a compound of general formula D;

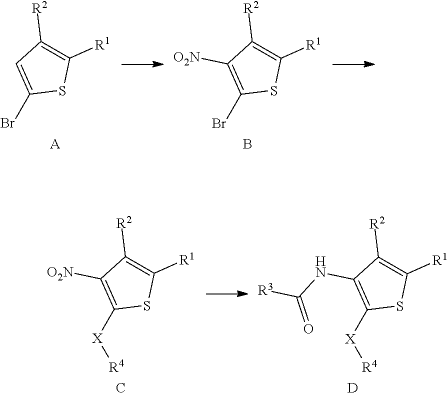

compound D is the compound of general formula (I), and R¹, R², R³, R⁴, and X are as described above.

Provided is a pharmaceutical composition comprising the compound of general formula (I) or the pharmaceutically acceptable salt thereof, and a pharmaceutically acceptable excipient.

Provided is use of the compound of general formula (I) or the pharmaceutically acceptable salt thereof, or the pharmaceutical composition in the preparation of a P2Y₁₄ receptor antagonist drug.

Provided is use of the compound of general formula (I) or the pharmaceutically acceptable salt thereof, or the pharmaceutical composition in the preparation of a drug for treating an inflammatory disease.

In the use, the drug is formulated into different dosage forms by adding pharmaceutically acceptable excipients.

The pharmaceutically acceptable excipient refers to various conventional excipients required for the preparation of different dosage forms, such as diluents, binders, disintegrants, glidants, lubricants, flavoring agents, inclusion materials, and adsorption materials, which are prepared into any common oral formulation by conventional preparation methods, such as granules, powders, tablets, capsules, pills, oral liquids, decoctions, and dripping pills.

Beneficial effects: Compared with the prior art, the present disclosure has the following advantages: The present disclosure discloses a class of thiophene-2-carboxylic acid derivatives with an inhibitory effect on a P2Y₁₄ receptor and pharmaceutically acceptable salts thereof, and such compounds have been proved by pharmacological experiments to have a significant inhibitory effect on the P2Y₁₄ receptor, and can be particularly used as drugs for treating inflammatory diseases.

### BRIEF DESCRIPTION OF THE DRAWING

FIG. 1 shows the relative expression of proteins downstream of the UDPG/P2Y₁₄ receptor signaling pathway in THP-I cells, wherein the data are represented by mean±standard deviation (n=4), and analyzed by one-way ANOVA (#### represents P<0.0001 compared with the normal group, * represents P<0.05 compared with the model control group, # represents P<0.01 compared with the model control group, *** represents P<0.001 compared with the model control group, and **** represents P<0.0001 compared with the model control group);

FIG. 2 shows the relative expression of proteins downstream of the UDPG/P2Y₁₄ receptor signaling pathway in THP-I cells, wherein the data are represented by mean±standard deviation (n=4), and analyzed by one-way ANOVA (#### represents P<0.0001 compared with the normal group, * represents P<0.05 compared with the model control group, # represents P<0.01 compared with the model control group, *** represents P<0.001 compared with the model control group, and **** represents P<0.0001 compared with the model control group);

FIG. 3 shows the relative expression of proteins downstream of the UDPG/P2Y₁₄ receptor signaling pathway in THP-I cells, wherein the data are represented by mean±standard deviation (n=4), and analyzed by one-way ANOVA (#### represents P<0.0001 compared with the normal group, * represents P<0.05 compared with the model control group, # represents P<0.01 compared with the model control group, *** represents P<0.001 compared with the model control group, and **** represents P<0.0001 compared with the model control group);

FIG. 4 shows the relative expression of proteins downstream of the UDPG/P2Y₁₄ receptor signaling pathway in THP-I cells, wherein the data are represented by mean±standard deviation (n=4), and analyzed by one-way ANOVA (#### represents P<0.0001 compared with the normal group, * represents P<0.05 compared with the model control group, # represents P<0.01 compared with the model control group, *** represents P<0.001 compared with the model control group, and **** represents P<0.0001 compared with the model control group);

FIG. 5 shows the level of IL-1β in the supernatant of the THP-I cell culture medium, wherein the data are represented by mean±standard deviation, and analyzed by one-way ANOVA (#### represents P<0.0001 compared with the normal group, * represents P<0.05 compared with the model control group, # represents P<0.01 compared with the model control group, *** represents P<0.001 compared with the model control group, and **** represents P<0.0001 compared with the model control group);

FIG. 6 shows the gene expression level of an inflammatory factor in peritoneal fluids from mice, wherein the data are represented by mean±standard deviation, and analyzed by one-way ANOVA (#### represents P<0.0001 compared with the normal group, * represents P<0.05 compared with the model control group, # represents P<0.01 compared with the model control group, *** represents P<0.001 compared with the model control group, and **** represents P<0.0001 compared with the model control group);

FIG. 7 shows the gene expression level of an inflammatory factor in peritoneal fluids from mice, wherein the data are represented by mean±standard deviation, and analyzed by one-way ANOVA (#### represents P<0.0001 compared with the normal group, * represents P<0.05 compared with the model control group, # represents P<0.01 compared with the model control group, *** represents P<0.001 compared with the model control group, and **** represents P<0.0001 compared with the model control group);

FIG. 8 shows the gene expression level of an inflammatory factor in peritoneal fluids from mice, wherein the data are represented by mean±standard deviation, and analyzed by one-way ANOVA (#### represents P<0.0001 compared with the normal group, * represents P<0.05 compared with the model control group, # represents P<0.01 compared with the model control group, *** represents P<0.001 compared with the model control group, and **** represents P<0.0001 compared with the model control group).

### DETAILED DESCRIPTION OF THE EMBODIMENTS

The content of the present disclosure will be specifically illustrated by the following examples. In the present disclosure, the following examples are intended to better illustrate the present disclosure and not to limit the scope of the present disclosure.

### Example 1 and Example 2

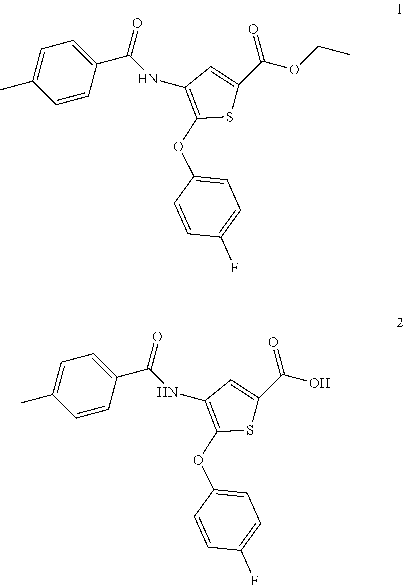

### Step 1: ethyl 5-bromo-4-nitrothiophene-2-carboxylate (1a)

Ethyl 5-bromothiophene-2-carboxylate (5.00 g, 21.27 mmol) was added dropwise to fuming nitric acid cooled to −20° C. in portions, with the temperature controlled not to be higher than −20° C. After the addition was completed, the mixture was reacted at this temperature for 2 h. After the reaction was completed as monitored by TLC, the reaction solution was added to ice water and extracted with ethyl acetate (3 times). The organic phases were combined, washed with saturated brine, and dried over anhydrous Na₂SO₄. The resulting mixture was purified by silica gel column chromatography (petroleum ether/ethyl acetate=50:1) to give a pale yellow solid (2.15 g, 36.09% yield). ¹H NMR (300 MHz, Chloroform-d) δ 8.15 (s, 1H), 4.38 (q, J=7.1 Hz, 2H), 1.39 (t, J=7.1 Hz, 3H).

### Step 2: ethyl 5-(4-fluorophenoxy)-4-nitrothiophene-2-carboxylate (1b)

Ethyl 5-bromo-4-nitrothiophene-2-carboxylate (0.20 g, 0.71 mmol) was dissolved in N,N-dimethylformamide (4 mL), and K₂CO₃ (0.20 g, 1-43 mmol) and 4-fluorophenol (0.12 g, 1.07 mmol) were added. The mixture was stirred at room temperature for 4 h. After the reaction was completed, water was added, and the mixture was extracted with ethyl acetate (3 times). The organic phases were combined, washed with saturated brine, and dried over anhydrous Na₂SO₄. The resulting mixture was purified by silica gel column chromatography (petroleum ether/ethyl acetate=20:1) to give a pale yellow solid (0.21 g, 94.48% yield). ¹H NMR (300 MHz, Chloroform-d) δ (ppm) 8.13 (s, 1H), 7.31 (dt, J=6.7, 2.1 Hz, 2H), 7.26-7.16 (m, 2H), 4.34 (q, J=7.1 Hz, 2H), 1.36 (t, J=7.1 Hz, 3H).

### Step 3: ethyl 5-(4-fluorophenoxy)-4-(4-methylbenzamido)thiophene-2-carboxylate (1)

Ethyl 5-(4-fluorophenoxy)-4-nitrothiophene-2-carboxylate (0.15 g, 0.48 mmol) was dissolved in a mixed solvent of ethanol/water (10/1) (5 mL), NH₄Cl (0.26 g, 4.82 mmol) was added, and iron powder (0.27 g, 4.82 mmol) was slowly added at 0° C. After the addition was completed, the mixture was stirred for 30 min and heated at reflux at 85° C. for 4 h. After the reaction was completed, the mixture was filtered through celite and concentrated under reduced pressure to remove the solvent, thereby giving a yellow oil.

The product obtained in the previous step was dissolved in a mixed solvent of anhydrous tetrahydrofuran (4 mL), and triethylamine (0.98 g, 0.97 mmol) and 4-methylbenzoyl chloride (0.97 g, 0.63 mmol) were slowly added dropwise at 0° C. After the addition was completed, the mixture was stirred at room temperature for 4 h. After the reaction was completed, water was added, and the mixture was extracted with ethyl acetate (3 times). The organic phases were combined, washed with saturated brine, and dried over anhydrous Na₂SO₄. The resulting mixture was purified by silica gel column chromatography (petroleum ether/ethyl acetate=8:1) to give a pale yellow solid (0.14 g, 73.46% yield). ¹H NMR (300 MHz, Chloroform-d) δ 8.53 (s, 1H), 7.85 (s, 1H), 7.75 (d, J=8.0 Hz, 2H), 7.33 (s, 3H), 7.23-7.08 (m, 4H), 4.37 (q, J=7.1 Hz, 2H), 2.46 (s, 3H), 1-40 (t, J=7.1 Hz, 3H).

### Step 4: 5-(4-fluorophenoxy)-4-(4-methylbenzamido)thiophene-2-carboxylic acid (2)

Ethyl 5-(4-fluorophenoxy)-4-(4-methylbenzamido)thiophene-2-carboxylate (100 mg, 0.253 mmol) was dissolved in a mixed solvent of methanol/tetrahydrofuran (1/1) (2 mL), and a 4 mol/L LiOH solution (1 mL) was added. After the addition was completed, the mixture was stirred at room temperature for 5 h. After the reaction was completed, 1 N HCl was added to adjust the pH of the solution to 2. The mixture was stirred for another 30 min and filtered to give a white solid (86 mg, 92.57% yield). ¹H NMR (300 MHz, DMSO-d₆) δ 10.10 (s, 1H), 7.81 (s, 1H), 7.77 (d, J=8.1 Hz, 2H), 7.29 (d, J=7.9 Hz, 2H), 7.27-7.21 (m, 4H), 2.36 (s, 3H).

### Example 3

The following compounds were prepared similarly as in Example 1 and Example 2:

| No. | Structural formula | Nuclear magnetic resonance data |
|---|---|---|
| 3-1 | 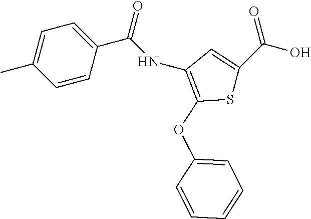 [SMILES: Cc1ccc(C(=O)Nc2cc(C(=O)O)sc2Oc2ccccc2)cc1] | ¹H NMR (300 MHz, DMSO-d₆) δ 10.13 (s, 1H), 7.89 (s, 1H), 7.80 (d, J = 8.1 Hz, 2H), 7.44 (dd, J = 8.5, 7.3 Hz, 2H), 7.30 (d, J = 7.9 Hz, 2H), 7.26-7.16 (m, 3H), 2.38 (s, 3H). |
| 3-2 | 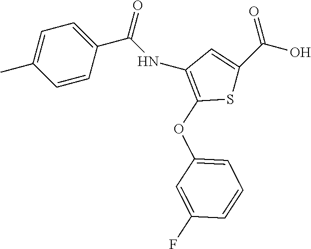 [SMILES: Cc1ccc(C(=O)Nc2cc(C(=O)O)sc2Oc2cccc(F)c2)cc1] | ¹H NMR (300 MHz, DMSO-d₆) δ 10.18 (s, 1H), 7.86 (s, 1H), 7.76 (d, J = 7.8 Hz, 2H), 7.44 (q, J = 7.6 Hz, 1H), 7.28 (d, J = 7.8 Hz, 2H), 7.11-6.97 (m, 3H), 2.35 (s, 3H). |
| 3-3 | 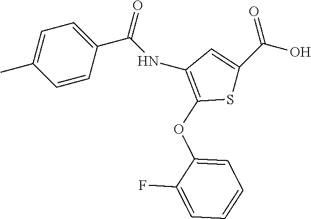 [SMILES: Cc1ccc(C(=O)Nc2cc(C(=O)O)sc2Oc2ccccc2F)cc1] | ¹H NMR (300 MHz, DMSO-d₆) δ 10.26 (s, 1H), 7.88 (s, 1H), 7.83 (d, J = 7.9 Hz, 2H), 7.49-7.39 (m, 1H), 7.32 (d, J = 8.1 Hz, 3H), 7.29-7.21 (m, 2H), 2.39 (s, 3H). |
| 3-4 | 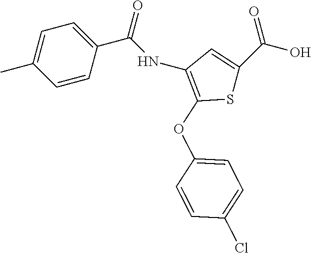 [SMILES: Cc1ccc(C(=O)Nc2cc(C(=O)O)sc2Oc2ccc(Cl)cc2)cc1] | ¹H NMR (300 MHz, DMSO-d₆) δ 10.13 (s, 1H), 7.84 (s, 1H), 7.74 (d, J = 8.1 Hz, 2H), 7.51-7.40 (m, 2H), 7.28 (d, J = 7.9 Hz, 2H), 7.24-7.14 (m, 2H), 2.36 (s, 3H). |
| 3-5 | 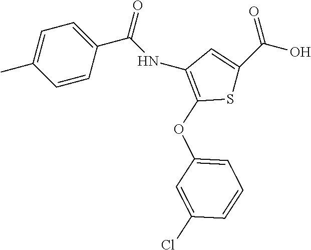 [SMILES: Cc1ccc(C(=O)Nc2cc(C(=O)O)sc2Oc2cccc(Cl)c2)cc1] | ¹H NMR (300 MHz, DMSO-d₆) δ 10.17 (s, 1H), 7.87 (s, 1H), 7.75 (d, J = 8.3 Hz, 2H), 7.44 (t, J = 8.4 Hz, 1H), 7.30 (d, J = 8.2 Hz, 2H), 7.28-7.23 (m, 2H), 7.20-7.10 (m, 1H), 2.37 (s, 3H). |
| 3-6 | 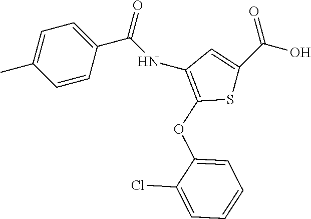 [SMILES: Cc1ccc(C(=O)Nc2cc(C(=O)O)sc2Oc2ccccc2Cl)cc1] | ¹H NMR (300 MHz, DMSO-d₆)8 10.24 (s, 1H), 7.87 (s, 1H), 7.82 (d, J = 7.9 Hz, 2H), 7.60 (dd, J = 7.9, 1.6 Hz, 1H), 7.46-7.34 (m, 1H), 7.33-7.20 (m, 4H), 2.37 (s, 3H). |
| 3-7 | 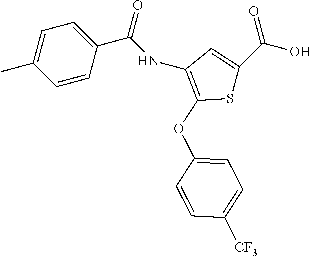 [SMILES: Cc1ccc(C(=O)Nc2cc(C(=O)O)sc2Oc2ccc(C(F)(F)F)cc2)cc1] | ¹H NMR (300 MHz, DMSO-d₆) δ 10.21 (s, 1H), 7.92 (s, 1H), 7.78 (d, J = 8.5 Hz, 2H), 7.73 (d, J = 7.9 Hz, 2H), 7.35 (d, J = 8.5 Hz, 2H), 7.28 (d, J = 7.9 Hz, 2H), 2.36 (s, 3H). |
| 3-8 | 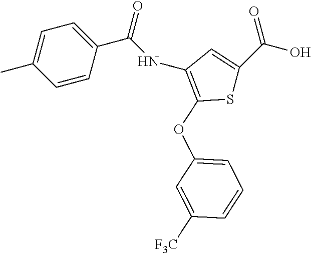 [SMILES: Cc1ccc(C(=O)Nc2cc(C(=O)O)sc2Oc2cccc(C(F)(F)F)c2)cc1] | ¹H NMR (300 MHz, DMSO-d₆) δ 13.34 (s, 1H), 10.16 (s, 1H), 7.87 (s, 1H), 7.70 (d, J = 8.2 Hz, 2H), 7.64 (t, J = 8.1 Hz, 1H), 7.54 (d, J = 7.8 Hz, 1H), 7.48 (d, J = 1.3 Hz, 2H), 7.27 (d, J = 8.0 Hz, 2H), 2.35 (s, 3H). |
| 3-9 | 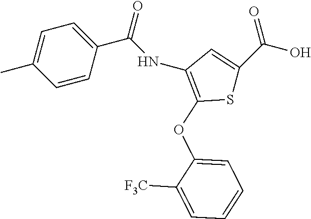 [SMILES: Cc1ccc(C(=O)Nc2cc(C(=O)O)sc2Oc2ccccc2C(F)(F)F)cc1] | ¹H NMR (300 MHz, DMSO-d₆) δ 10.18 (s, 1H), 7.85 (s, 1H), 7.82-7.70 (m, 4H), 7.45-7.35 (m, 2H), 7.31 (d, J = 8.0 Hz, 2H), 2.37 (s, 3H). |
| 3-10 | 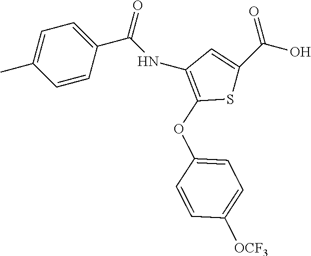 [SMILES: Cc1ccc(C(=O)Nc2cc(C(=O)O)sc2Oc2ccc(OC(F)(F)F)cc2)cc1] | ¹H NMR (300 MHz, DMSO-d₆) δ 10.16 (s, 1H), 7.87 (s, 1H), 7.73 (d, J = 8.2 Hz, 2H), 7.43 (d, J = 8.7 Hz, 2H), 7.32-7.25 (m, 4H), 2.37 (s, 3H). |
| 3-11 | 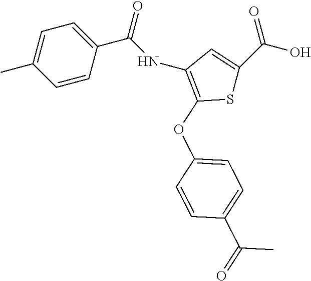 [SMILES: CC(=O)c1ccc(Oc2sc(C(=O)O)cc2NC(=O)c2ccc(C)cc2)cc1] | ¹H NMR (300 MHz, DMSO-d₆) δ 10.19 (s, 1H), 8.01 (d, J = 8.9 Hz, 2H), 7.90 (s, 1H), 7.73 (d, J = 8.1 Hz, 2H), 7.32-7.20 (m, 4H), 2.55 (s, 3H), 2.36 (s, 3H). |
| 3-12 | 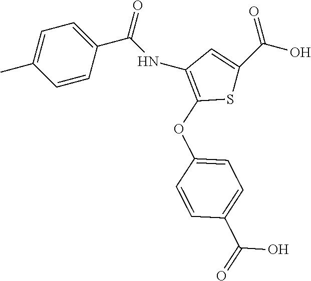 [SMILES: Cc1ccc(C(=O)Nc2cc(C(=O)O)sc2Oc2ccc(C(=O)O)cc2)cc1] | ¹H NMR (300 MHz, DMSO-d₆) δ 13.15 (s, 2H), 10.20 (s, 1H), 7.98 (d, J = 8.8 Hz, 2H), 7.92 (s, 1H), 7.73 (d, J = 7.9 Hz, 2H), 7.27 (d, J = 7.9 Hz, 2H), 7.24 (d, J = 8.8 Hz, 2H), 2.35 (s, 3H). |
| 3-13 | 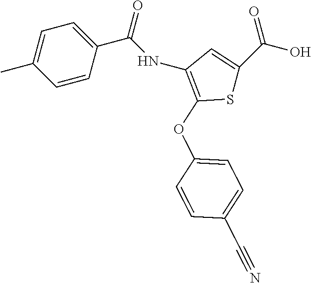 [SMILES: Cc1ccc(C(=O)Nc2cc(C(=O)O)sc2Oc2ccc(C#N)cc2)cc1] | ¹H NMR (300 MHz, DMSO-d₆) δ 13.33 (s, 1H), 10.17 (s, 1H), 7.89 (s, 1H), 7.87 (d, J = 8.2 Hz, 2H), 7.69 (d, J = 7.9 Hz, 2H), 7.32-7.21 (m, 4H), 2.34 (s, 3H). |
| 3-14 | 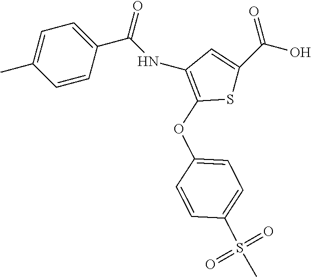 [SMILES: Cc1ccc(C(=O)Nc2cc(C(=O)O)sc2Oc2ccc(S(C)(=O)=O)cc2)cc1] | ¹H NMR (300 MHz, DMSO-d₆) δ 10.22 (s, 1H), 7.96 (d, J = 8.8 Hz, 2H), 7.92 (s, 1H), 7.72 (d, J = 8.0 Hz, 2H), 7.37 (d, J = 8.9 Hz, 2H), 7.28 (d, J = 7.9 Hz, 2H), 3.20 (s, 3H), 2.36 (s, 3H). |
| 3-15 | 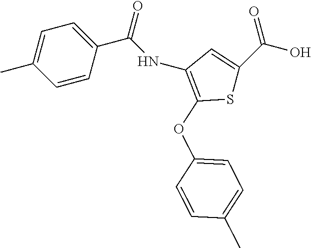 [SMILES: Cc1ccc(Oc2sc(C(=O)O)cc2NC(=O)c2ccc(C)cc2)cc1] | ¹H NMR (300 MHz, DMSO-d₆) δ 10.11 (s, 1H), 7.87 (s, 1H), 7.82 (d, J = 8.0 Hz, 2H), 7.31 (d, J = 8.0 Hz, 2H), 7.24 (d, J = 8.5 Hz, 2H), 7.12 (d, J = 8.6 Hz, 2H), 2.38 (s, 3H), 2.30 (s, 3H). |
| 3-16 | 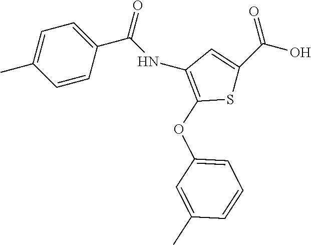 [SMILES: Cc1ccc(C(=O)Nc2cc(C(=O)O)sc2Oc2cccc(C)c2)cc1] | ¹H NMR (300 MHz, DMSO-d₆) δ 10.09 (s, 1H), 7.82 (s, 1H), 7.76 (d, J = 8.1 Hz, 2H), 7.27 (dd, J = 8.0, 3.2 Hz, 3H), 7.05-6.92 (m, 3H), 2.35 (s, 3H), 2.29 (s, 3H). |
| 3-17 | 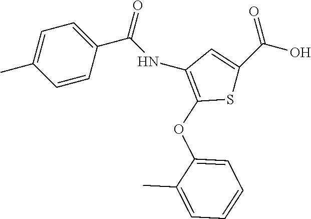 [SMILES: Cc1ccc(C(=O)Nc2cc(C(=O)O)sc2Oc2ccccc2C)cc1] | ¹H NMR (300 MHz, DMSO-d₆) δ 10.19 (s, 1H), 7.93- 7.85 (m, 3H), 7.37 (dd, J = 7.8, 5.6 Hz, 3H), 7.34- 7.27 (m, 1H), 7.27-7.15 (m, 2H), 2.43 (s, 3H), 2.35 (s, 3H). |
| 3-18 | 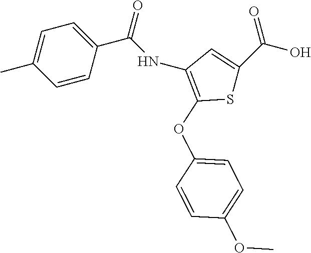 [SMILES: COc1ccc(Oc2sc(C(=O)O)cc2NC(=O)c2ccc(C)cc2)cc1] | ¹H NMR (300 MHz, DMSO-d₆) δ 10.16 (s, 1H), 7.86 (d, J = 7.9 Hz, 2H), 7.85 (s, 1H), 7.32 (d, J = 7.9 Hz, 2H), 7.29-7.14 (m, 2H), 7.07-6.95 (m, 2H), 3.77 (s, 3H), 2.39 (s, 3H). |
| 3-19 | 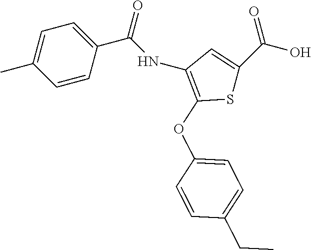 [SMILES: CCc1ccc(Oc2sc(C(=O)O)cc2NC(=O)c2ccc(C)cc2)cc1] | ¹H NMR (300 MHz, DMSO-d₆) δ 10.12 (s, 1H), 7.84 (s, 1H), 7.79 (d, J = 8.1 Hz, 2H), 7.27 (d, J = 8.1 Hz, 2H), 7.23 (d, J = 8.5 Hz, 2H), 7.11 (d, J = 8.6 Hz, 2H), 2.57 (q, J = 7.6 Hz, 2H), 2.34 (s, 3H), 1.14 (t, J = 7.6 Hz, 3H). |
| 3-20 | 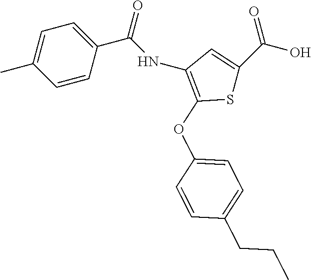 [SMILES: CCCc1ccc(Oc2sc(C(=O)O)cc2NC(=O)c2ccc(C)cc2)cc1] | ¹H NMR (300 MHz, DMSO-d₆) δ 13.12 (s, 1H), 10.06 (s, 1H), 7.82 (s, 1H), 7.76 (d, J = 8.1 Hz, 2H), 7.32-7.16 (m, 4H), 7.15-7.04 (m, 2H), 2.58-2.51 (m, 2H), 2.35 (s, 3H), 1.55 (h, J = 7.3 Hz, 2H), 0.85 (t, J = 7.3 Hz, 3H). |
| 3-21 | 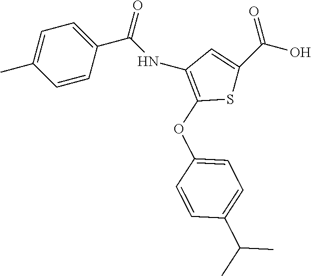 [SMILES: Cc1ccc(C(=O)Nc2cc(C(=O)O)sc2Oc2ccc(C(C)C)cc2)cc1] | ¹H NMR (300 MHz, DMSO-d₆) δ 10.09 (s, 1H), 7.85 (s, 1H), 7.78 (d, J = 8.2 Hz, 2H), 7.28 (dd, J = 8.4, 1.6 Hz, 4H), 7.12 (d, J = 8.6 Hz, 2H), 2.89 (h, J = 6.9 Hz, 1H), 2.36 (s, 3H), 1.17 (d, J = 6.9 Hz, 6H). |
| 3-22 | 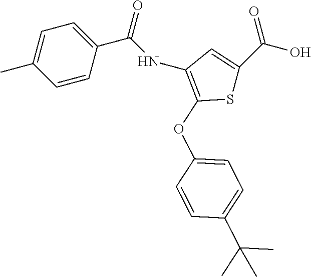 [SMILES: Cc1ccc(C(=O)Nc2cc(C(=O)O)sc2Oc2ccc(C(C)(C)C)cc2)cc1] | ¹H NMR (300 MHz, DMSO-d₆) δ 10.07 (s, 1H), 7.84 (s, 1H), 7.76 (d, J = 7.8 Hz, 2H), 7.41 (d, J = 8.4 Hz, 2H), 7.27 (d, J = 7.8 Hz, 2H), 7.11 (d, J = 8.4 Hz, 2H), 2.34 (s, 3H), 1.25 (s, 9H). |
| 3-23 | 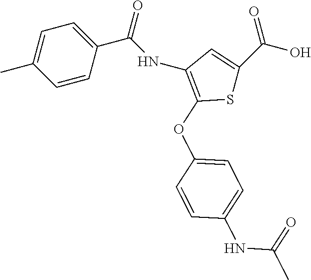 [SMILES: CC(=O)Nc1ccc(Oc2sc(C(=O)O)cc2NC(=O)c2ccc(C)cc2)cc1] | ¹H NMR (300 MHz, DMSO-d₆) δ 10.12 (s, 1H), 10.05 (s, 1H), 7.84 (s, 1H), 7.82 (d, J = 8.2 Hz, 2H), 7.63 (d, J = 9.0 Hz, 2H), 7.31 (d, J = 7.9 Hz, 2H), 7.18 (d, J = 9.0 Hz, 2H), 2.38 (s, 3H), 2.05 (s, 3H). |
| 3-24 | 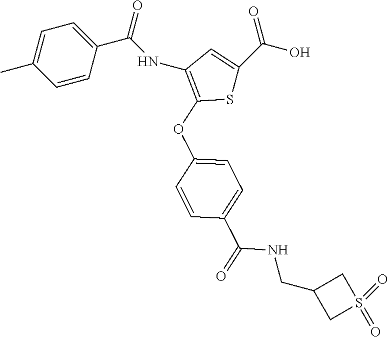 [SMILES: Cc1ccc(C(=O)Nc2cc(C(=O)O)sc2Oc2ccc(C(=O)NCC3CS(=O)(=O)C3)cc2)cc1] | ¹H NMR (300 MHz, DMSO-d₆) δ 10.12 (s, 1H), 10.05 (s, 1H), 7.84 (s, 1H), 7.82 (d, J = 8.2 Hz, 2H), 7.63 (d, J = 9.0 Hz, 2H), 7.31 (d, J = 7.9 Hz, 2H), 7.18 (d, J = 9.0 Hz, 2H),4.01 (dd,J = 13.5, 6.0 Hz, 2H), 3.80 (dd, J = 13.5, 6.1 Hz, 2H), 3.74-3.62 (m, 1H), 3.43(1,J = 5.3 Hz, 2H), 2.39 (s, 3H). |
| 3-25 | 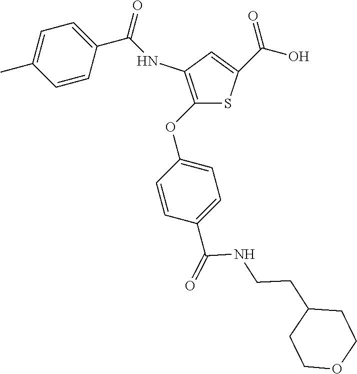 [SMILES: Cc1ccc(C(=O)Nc2cc(C(=O)O)sc2Oc2ccc(C(=O)NCCC3CCOCC3)cc2)cc1] | ¹H NMR (300 MHz, DMSO-d₆) δ 10.12 (s, 1H), 10.05 (s, 1H), 7.84 (s, 1H), 7.82 (d, J = 8.2 Hz, 2H), 7.63 (d, J = 9.0 Hz, 2H), 7.31 (d, J = 7.9 Hz, 2H), 7.18 (d, J = 9.0 Hz, 2H), 3.82 (ddd, J = 11.9, 7.1, 5.0 Hz, 2H), 3.74-3.62 (m, 1H),3.38 (ddd,J = 11.6, 7.4,4.9 Hz, 2H), 3.29 (td,J = 5.7, 4.4 Hz, 2H), 2.39 (s, 3H), 1.65 (dddd, J = 13.5, 7.3, 6.3, 5.0 Hz, 2H), 1.53 (q, J = 5.8 Hz, 2H). |

### Example 4 and Example 5

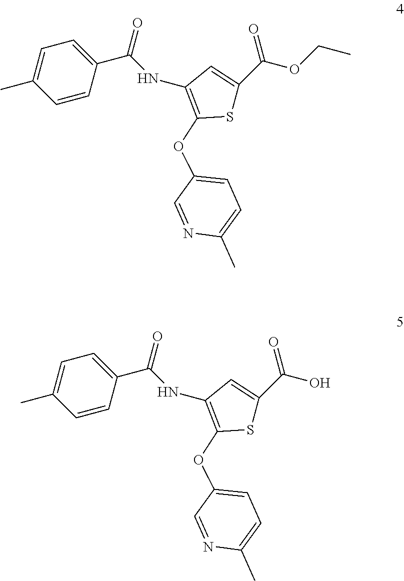

### Step 1: Ethyl 5-((6-methylpyridin-3-yl)oxy)-4-nitrothiophene-2-carboxylate (4b)

Ethyl 5-bromo-4-nitrothiophene-2-carboxylate (0.20 g, 0.71 mmol) was dissolved in N,N-dimethylformamide (4 mL), and K₂CO₃ (0.20 g, 1-43 mmol) and 3-hydroxy-6-methylpyridine (0.12 g, 1.07 mmol) were added. The mixture was stirred at room temperature for 4 h. After the reaction was completed, water was added, and the mixture was extracted with ethyl acetate (3 times). The organic phases were combined, washed with saturated brine, and dried over anhydrous Na₂SO₄. The resulting mixture was purified by silica gel column chromatography (petroleum ether/ethyl acetate=20:1) to give a pale yellow solid (0.21 g, 94.48% yield). ¹H NMR (300 MHz, DMSO-d₆) δ 8.65 (d, J=2.9 Hz, 1H), 8.07 (s, 1H), 7.92 (dd, J=8.6, 3.0 Hz, 1H), 7.47 (d, J=8.6 Hz, 1H), 4.27 (q, J=7.1 Hz, 2H), 2.55 (s, 3H), 1.25 (t, J=7.1 Hz, 3H).

### Step 2: ethyl 5-((6-methylpyridin-3-yl)oxy)-4-(4-methylbenzamido)thiophene-2-carboxylate (4)

Ethyl 5-((6-methylpyridin-3-yl)oxy)-4-nitrothiophene-2-carboxylate (0.15 g, 0.49 mmol) was dissolved in a mixed solvent of ethanol/water (10/1) (5 mL), NH₄Cl (0.26 g, 4.87 mmol) was added, and iron powder (0.27 g, 4.87 mmol) was slowly added at 0° C. After the addition was completed, the mixture was stirred for 30 min and heated at reflux at 85° C. for 4 h. After the reaction was completed, the mixture was filtered through celite and concentrated under reduced pressure to remove the solvent, thereby giving a yellow oil.

The product obtained in the previous step was dissolved in a mixed solvent of anhydrous tetrahydrofuran (4 mL), and triethylamine (0.98 g, 0.97 mmol) and 4-methylbenzoyl chloride (0.98 g, 0.63 mmol) were slowly added dropwise at 0° C. After the addition was completed, the mixture was stirred at room temperature for 4 h. After the reaction was completed, water was added, and the mixture was extracted with ethyl acetate (3 times). The organic phases were combined, washed with saturated brine, and dried over anhydrous Na₂SO₄. The resulting mixture was purified by silica gel column chromatography (petroleum ether/ethyl acetate=8:1) to give a pale yellow solid (0.13 g, 67.4% yield). ¹H NMR (300 MHz, DMSO-d₆) δ 10.16 (s, 1H), 8.38 (d, J=3.0 Hz, 1H), 7.92 (s, 1H), 7.76 (d, J=7.9 Hz, 2H), 7.53 (dd, J=8.6, 3.0 Hz, 1H), 7.29 (dd, J=8.4, 2.3 Hz, 3H), 4.27 (q, J=7.1 Hz, 2H), 2.45 (s, 3H), 2.36 (s, 3H), 1.28 (t, J=7.1 Hz, 3H).

### Step 3: 5-((6-methylpyridin-3-yl)oxy)-4-(4-methylbenzamido)thiophene-2-carboxylic acid (5)

Ethyl 5-(4-fluorophenoxy)-4-(4-methylbenzamido)thiophene-2-carboxylate (100.00 mg, 0.253 mmol) was dissolved in a mixed solvent of methanol/tetrahydrofuran (1/1) (2 mL), and a 4 mol/L LiOH solution (1 mL) was added. After the addition was completed, the mixture was stirred at room temperature for 5 h. After the reaction was completed, 1 N HCl was added to adjust the pH of the solution to 5-7. The mixture was stirred for another 30 min and filtered to give a white solid (75.5 mg, 77.75% yield). 1H NMR (300 MHz, DMSO-d₆) δ 10.13 (s, 1H), 8.37 (d, J=3.0 Hz, 1H), 7.85 (s, 1H), 7.76 (d, J=7.8 Hz, 2H), 7.51 (dd, J=8.6, 3.0 Hz, 1H), 7.28 (d, J=8.1 Hz, 3H), 2.43 (s, 3H), 2.35 (s, 3H).

### Example 6

The following compounds were prepared similarly as in Example 4 and Example 5:

| No. | Structural formula | Nuclear magnetic resonance data |
|---|---|---|
| 6-1 | 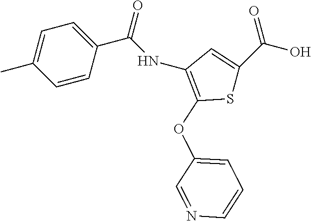 [SMILES: Cc1ccc(C(=O)Nc2cc(C(=O)O)sc2Oc2cccnc2)cc1] | ¹H NMR (300 MHz, DMSO-d₆) δ 13.32 (s, 1H), 10.18 (s, 1H), 8.51 (d, J = 2.9 Hz, 1H), 8.39 (dd, J = 4.6, 1.3 Hz, 1H), 7.86 (s, 1H), 7.73 (d, J = 8.2 Hz, 2H), 7.59 (ddd, J = 8.5, 3.0, 1.3 Hz, 1H), 7.44 (dd, J = 8.5, 4.6 Hz, 1H), 7.28 (d, J = 8.0 Hz, 2H), 2.35 (s, 3H). |
| 6-2 | 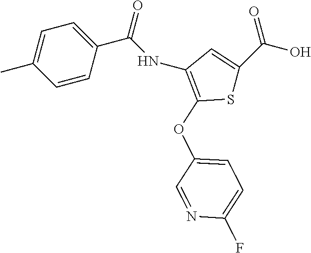 [SMILES: Cc1ccc(C(=O)Nc2cc(C(=O)O)sc2Oc2ccc(F)nc2)cc1] | ¹H NMR (300 MHz, DMSO-d₆) δ 10.19 (s, 1H), 8.17 (dd, J = 3.2, 1.6 Hz, 1H), 7.91-7.80 (m, 2H), 7.76 (d, J = 8.0 Hz, 2H), 7.31 (d, J = 8.0 Hz, 2H), 7.26 (dd, J = 8.9, 3.3 Hz, 1H), 2.38 (s, 3H). |
| 6-3 | 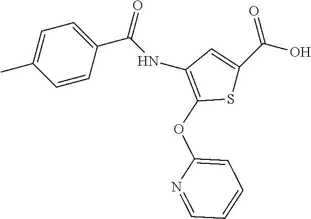 [SMILES: Cc1ccc(C(=O)Nc2cc(C(=O)O)sc2Oc2ccccn2)cc1] | ¹H NMR (300 MHz, DMSO-d₆) δ 10.17 (s, 1H), 7.85 (s, 1H), 7.74 (d, J = 7.8 Hz, 2H), 7.60 (d, J = 7.0 Hz, 1H), 7.47 (t, J = 8.1 Hz, 1H), 7.29 (d, J = 7.8 Hz, 2H), 6.53 (d, J = 9.3 Hz, 1H), 6.31 (t, J = 6.8 Hz, 1H), 2.36 (s, 3H) |
| 6-4 | 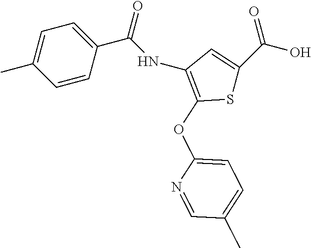 [SMILES: Cc1ccc(C(=O)Nc2cc(C(=O)O)sc2Oc2ccc(C)cn2)cc1] | ¹H NMR (300 MHz, DMSO-d₆) δ 10.13 (s, 1H), 8.37 (d, J = 3.0 Hz, 1H), 7.85 (s, 1H), 7.76 (d, J = 7.8 Hz, 2H), 7.51 (dd, J = 8.6, 3.0 Hz, 1H), 7.28 (d, J = 8.1 Hz, 3H), 2.43 (s, 3H), 2.35 (s, 3H). |
| 6-5 | 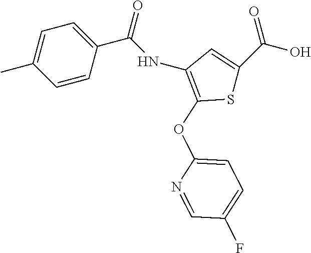 [SMILES: Cc1ccc(C(=O)Nc2cc(C(=O)O)sc2Oc2ccc(F)cn2)cc1] | ¹H NMR (300 MHz, DMSO-d₆) δ 10.09 (s, 1H), 8.33 (d, J = 3.1 Hz, 1H), 7.98-7.87 (m, 2H), 7.82 (d, J = 8.1 Hz, 2H), 7.30 (td, J = 6.5, 3.1 Hz, 3H), 2.37 (s, 3H). |
| 6-6 | 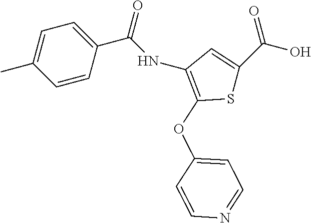 [SMILES: Cc1ccc(C(=O)Nc2cc(C(=O)O)sc2Oc2ccncc2)cc1] | ¹H NMR (300 MHz, DMSO-d₆) δ 10.35 (s, 1H), 7.85 (s, 1H), 7.77 (dd, J = 8.0, 6.3 Hz, 4H), 7.32 (d, J = 7.9 Hz, 2H), 6.18 (d, J = 7.7 Hz, 2H), 2.37 (s, 3H). |
| 6-7 | 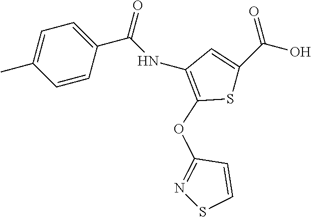 [SMILES: Cc1ccc(C(=O)Nc2cc(C(=O)O)sc2Oc2ccsn2)cc1] | ¹H NMR (300 MHz, DMSO-d₆) δ 10.09 (s, 1H), 9.03 (d, J = 4.8 Hz, 1H), 7.82 (s, 1H), 7.80 (d, J = 7.6 Hz, 2H), 7.28 (d, J = 7.9 Hz, 2H), 7.05 (d, J = 4.8 Hz, 1H), 2.34 (s, 3H). |

### Example 7 and Example 8

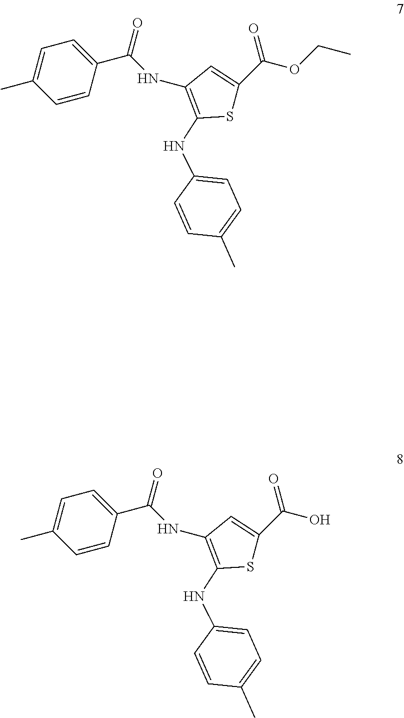

### Step 1: ethyl 4-nitro-5-(p-tolylamino)thiophene-2-carboxylate (7b)

Ethyl 5-bromo-4-nitrothiophene-2-carboxylate (0.15 g, 0.53 mmol) was dissolved in toluene (3 mL), and palladium acetate (3.61 mg, 0.016 mmol), BINAP (13.34 mg, 0.021 mmol), and cesium carbonate (0.244 g, 0.750 mmol) were added. The mixture was heated to 115° C. under argon atmosphere. After 5 min 4-methylaniline (0.069 g, 0.643 mmol) was added, and the mixture was reacted for 4 h. After the reaction was completed, the mixture was concentrated under reduced pressure to remove the organic solvent, and the residue was purified by silica gel column chromatography (petroleum ether/ethyl acetate=15:1) to give a pale yellow solid (0.92 g, 56.09% yield). ¹H NMR (300 MHz, DMSO-d₆) δ 10.76 (s, 1H), 7.88 (s, 1H), 7.43-7.37 (m, 2H), 7.31 (d, J=8.4 Hz, 2H), 4.21 (q, J=7.1 Hz, 2H), 2.35 (s, 3H), 1.23 (t, J=7.1 Hz, 3H).

### Step 2: ethyl 4-(4-methylbenzamido)-5-(p-tolylamino)thiophene-2-carboxylate (7)

Ethyl 4-nitro-5-(p-tolylamino)thiophene-2-carboxylate (0.15 g, 0.49 mmol) was dissolved in a mixed solvent of ethanol/water (10/1) (5 mL), NH₄Cl (0.26 g, 4.90 mmol) was added, and iron powder (0.27 g, 4.90 mmol) was slowly added at 0° C. After the addition was completed, the mixture was stirred for 30 min and heated at reflux at 85° C. for 4 h. After the reaction was completed, the mixture was filtered through celite and concentrated under reduced pressure to remove the solvent, thereby giving a yellow oil.

The product obtained in the previous step was dissolved in a mixed solvent of anhydrous tetrahydrofuran (4 mL), and triethylamine (0.99 g, 0.99 mmol) and 4-methylbenzoyl chloride (0.98 g, 0.64 mmol) were slowly added dropwise at 0° C. After the addition was completed, the mixture was stirred at room temperature for 4 h. After the reaction was completed, water was added, and the mixture was extracted with ethyl acetate (3 times). The organic phases were combined, washed with saturated brine, and dried over anhydrous Na₂SO₄. The resulting mixture was purified by silica gel column chromatography (petroleum ether/ethyl acetate=8:1) to give a pale yellow solid (0.12 g, 62.0% yield). ¹H NMR (300 MHz, DMSO-d₆) δ 9.82 (s, 1H), 8.69 (s, 1H), 7.87 (d, J=8.1 Hz, 2H), 7.79 (s, 1H), 7.32 (d, J=8.0 Hz, 2H), 7.13 (s, 4H), 4.23 (q, J=7.1 Hz, 2H), 2.38 (s, 3H), 2.25 (s, 3H), 1.27 (t, J=7.1 Hz, 4H).

### Step 3: 4-(4-methylbenzamido)-5-(p-tolylamino)thiophene-2-carboxylic acid (8)

Ethyl 4-(4-methylbenzamido)-5-(p-tolylamino)thiophene-2-carboxylate (100.00 mg, 0.253 mmol) was dissolved in a mixed solvent of methanol/tetrahydrofuran (1/1) (2 mL), and a 4 mol/L LiOH solution (1 mL) was added. After the addition was completed, the mixture was stirred at room temperature for 5 h. After the reaction was completed, 1 N HCl was added to adjust the pH of the solution to 5-7. The mixture was stirred for another 30 min and filtered to give a white solid (45.5 mg, 49.0% yield). ¹H NMR (300 MHz, DMSO-d₆) δ 11.90 (s, 1H), 10.28 (s, 1H), 7.78 (d, J=7.4 Hz, 2H), 7.53 (d, J=8.2 Hz, 2H), 7.39 (d, J=7.5 Hz, 2H), 7.11 (d, J=8.2 Hz, 2H), 2.40 (s, 3H), 2.25 (s, 3H).

### Example 9

The following compounds were prepared similarly as in Example 7 and Example 8:

| No. | Structural formula | Nuclear magnetic resonance data |
|---|---|---|
| 9-1 | 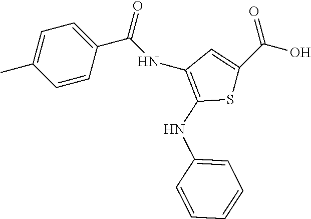 [SMILES: Cc1ccc(C(=O)Nc2cc(C(=O)O)sc2Nc2ccccc2)cc1] | ¹H NMR (300 MHz, DMSO-d₆) δ 11.91 (s, 1H), 10.27 (s, 1H), 7.78 (d, J = 7.4 Hz, 2H), 7.53 (d, J = 8.2 Hz, 2H), 7.42 (d, J = 7.5 Hz, 2H), 7.29-7.19 (m, 3H), 2.40 (s, 3H). |
| 9-2 | 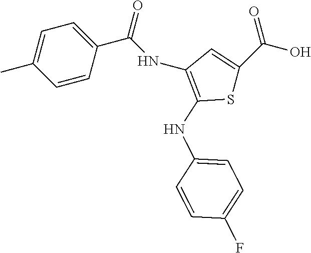 [SMILES: Cc1ccc(C(=O)Nc2cc(C(=O)O)sc2Nc2ccc(F)cc2)cc1] | ¹H NMR (300 MHz, DMSO-d₆) δ 11.90 (s, 1H), 10.29 (s, 1H), 7.78 (d, J = 7.4 Hz, 2H), 7.69 (d, J = 8.2 Hz, 2H), 7.39 (d, J = 7.5 Hz, 2H), 7.28 (d, J = 8.3 Hz, 2H), 2.42 (s, 3H). |
| 9-3 | 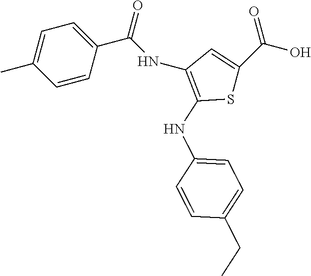 [SMILES: CCc1ccc(Nc2sc(C(=O)O)cc2NC(=O)c2ccc(C)cc2)cc1] | ¹H NMR (300 MHz, DMSO-d₆) δ 11.90 (s, 1H), 10.28 (s, 1H), 7.78 (d, J = 7.4 Hz, 2H), 7.53 (d, J = 8.2 Hz, 2H), 7.39 (d, J = 7.5 Hz, 2H), 7.11 (d, J = 8.2 Hz, 2H), 2.57 (q, J = 7.6 Hz, 2H), 2.40 (s, 3H), 1.14 (t, J = 7.6 Hz, 3H). |
| 9-4 | 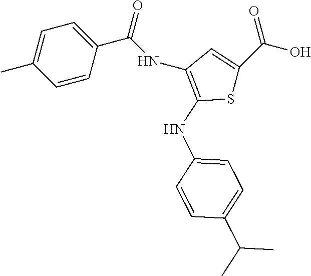 [SMILES: Cc1ccc(C(=O)Nc2cc(C(=O)O)sc2Nc2ccc(C(C)C)cc2)cc1] | ¹H NMR (300 MHz, DMSO-d₆) δ 11.90 (s, 1H), 10.28 (s, 1H), 7.78 (d, J = 7.4 Hz, 2H), 7.53 (d, J = 8.2 Hz, 2H), 7.39 (d, J = 7.5 Hz, 2H), 7.11 (d, J = 8.2 Hz, 2H), 2.89 (h, J = 6.9 Hz. 6H). |

### Example 10 and Example 11

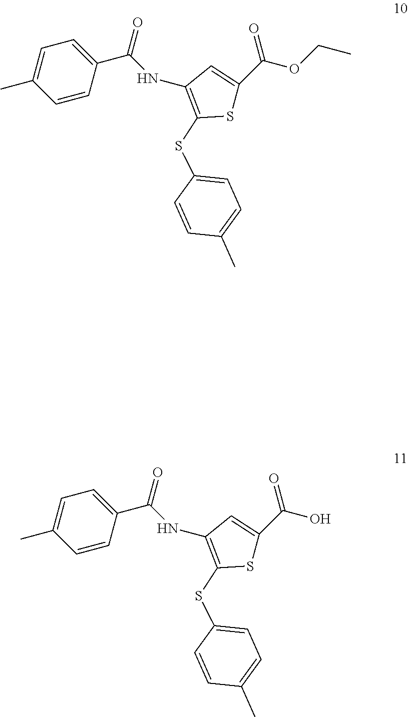

### Step 1: ethyl 4-nitro-5-(p-tolylthio)thiophene-2-carboxylate (7b)

Ethyl 5-bromo-4-nitrothiophene-2-carboxylate (0.20 g, 0.71 mmol) was dissolved in N,N-dimethylformamide (4 mL), and K₂CO₃ (0.20 g, 1-43 mmol) and 4-methylthiophenol (0.13 g, 1.07 mmol) were added. The mixture was stirred at room temperature for 4 h. After the reaction was completed, water was added, and the mixture was extracted with ethyl acetate (3 times). The organic phases were combined, washed with saturated brine, and dried over anhydrous Na₂SO₄. The resulting mixture was purified by silica gel column chromatography (petroleum ether/ethyl acetate=20:1) to give a yellow solid (0.16 g, 73.2% yield). ¹H NMR (300 MHz, DMSO-d₆) δ 8.10 (s, 1H), 7.74-7.64 (m, 2H), 7.51-7.42 (m, 2H), 4.24 (q, J=7.2 Hz, 2H), 2.43 (s, 3H), 1.23 (t, J=7.1 Hz, 3H).

### Step 2: ethyl 4-(4-methylbenzamido)-5-(p-tolylthio)thiophene-2-carboxylate (7)

Ethyl 4-nitro-5-(p-tolylthio)thiophene-2-carboxylate (0.15 g, 0.46 mmol) was dissolved in a mixed solvent of ethanol/water (10/1) (5 mL), NH₄Cl (0.25 g, 4.64 mmol) was added, and iron powder (0.26 g, 4.64 mmol) was slowly added at 0° C. After the addition was completed, the mixture was stirred for 30 min and heated at reflux at 85° C. for 4 h. After the reaction was completed, the mixture was filtered through celite and concentrated under reduced pressure to remove the solvent, thereby giving a yellow oil.

The product obtained in the previous step was dissolved in a mixed solvent of anhydrous tetrahydrofuran (4 mL), and triethylamine (0.94 g, 0.93 mmol) and 4-methylbenzoyl chloride (0.94 g, 0.60 mmol) were slowly added dropwise at 0° C. After the addition was completed, the mixture was stirred at room temperature for 4 h. After the reaction was completed, water was added, and the mixture was extracted with ethyl acetate (3 times). The organic phases were combined, washed with saturated brine, and dried over anhydrous Na₂SO₄. The resulting mixture was purified by silica gel column chromatography (petroleum ether/ethyl acetate=8:1) to give a pale yellow solid (0.11 g, 57.6% yield). 1H NMR (300 MHz, DMSO-d₆) δ 10.14 (s, 1H), 7.99 (s, 1H), 7.82 (d, J= 8.3 Hz, 2H), 7.33 (d, J=7.8 Hz, 2H), 7.27 (d, J=8.3 Hz, 2H), 7.19 (d, J=8.4 Hz, 2H), 4.27 (q, J=7.2 Hz, 2H), 2.38 (s, 3H), 2.27 (s, 3H), 1.27 (t, J=7.1 Hz, 3H).

### Step 3: 4-(4-methylbenzamido)-5-(p-tolylthio)thiophene-2-carboxylic acid (8)

Ethyl 4-(4-methylbenzamido)-5-(p-tolylamino)thiophene-2-carboxylate (100.00 mg, 0.253 mmol) was dissolved in a mixed solvent of methanol/tetrahydrofuran (1/1) (2 mL), and a 4 mol/L LiOH solution (1 mL) was added. After the addition was completed, the mixture was stirred at room temperature for 5 h. After the reaction was completed, 1 N HCl was added to adjust the pH of the solution to 4. The mixture was stirred for another 30 min and filtered to give a white solid (88.7 mg, 95.0% yield). ¹H NMR (300 MHz, DMSO-d₆) δ 10.10 (s, 1H), 7.92 (s, 1H), 7.83 (d, J=8.2 Hz, 2H), 7.31 (d, J=8.2 Hz, 2H), 7.23 (d, J=8.2 Hz, 2H), 7.16 (d, J=8.4 Hz, 2H), 2.36 (s, 3H), 2.25 (s, 3H).

### Example 12

The following compounds were prepared similarly as in Example 10 and Example 11:

| No. | Structural formula | Nuclear magnetic resonance data |
|---|---|---|
| 12-1 | 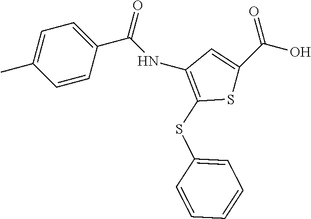 [SMILES: Cc1ccc(C(=O)Nc2cc(C(=O)O)sc2Sc2ccccc2)cc1] | ¹H NMR (300 MHz, DMSO-d₆) δ10.07 (s, 1H), 7.92 (d, J = 7.9 Hz, 2H), 7.53 (d, J = 8.2 Hz, 2H), 7.42 (d, J = 7.9 Hz, 2H), 7.31-7.20 (m, 3H), 2.39 (s, 3H). |
| 12-2 | 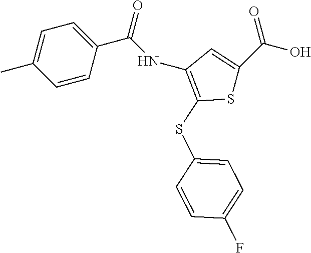 [SMILES: Cc1ccc(C(=O)Nc2cc(C(=O)O)sc2Sc2ccc(F)cc2)cc1] | ¹H NMR (300 MHz, DMSO-d₆) δ 10.08 (s, 1H), 7.92 (d, J = 7.9 Hz, 2H), 7.53 (d, J = 8.2 Hz, 2H), 7.42 (d, J = 7.9 Hz, 2H), 7.28 (d, J = 8.3 Hz, 2H), 2.39 (s, 3H). |
| 12-3 | 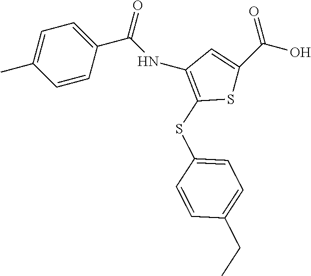 [SMILES: CCc1ccc(Sc2sc(C(=O)O)cc2NC(=O)c2ccc(C)cc2)cc1] | ¹H NMR (300 MHz, DMSO-d₆) δ10.09 (s, 1H), 7.92 (d, J = 7.9 Hz, 2H), 7.53 (d, J = 8.2 Hz, 2H), 7.42 (d, J = 7.9 Hz, 2H), 7.28 (d, J = 8.3 Hz, 2H), 2.57 (q, J = 7.6 Hz, 2H), 2.40 (s, 3H), 1.14 (t, J = 7.6 Hz, 3H). |
| 12-4 | 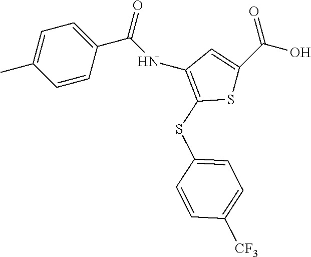 [SMILES: Cc1ccc(C(=O)Nc2cc(C(=O)O)sc2Sc2ccc(C(F)(F)F)cc2)cc1] | ¹H NMR (300 MHz, DMSO-d₆) δ10.08 (s, 1H), 7.98 (d, J = 8.2 Hz, 2H), 7.64 (d, J = 7.3 Hz, 2H), 7.51 (d, J = 8.2 Hz, 2H), 7.40 (d, J = 7.3 Hz, 2H), 2.39 (s, 3H). |

### Example 13 and Example 14

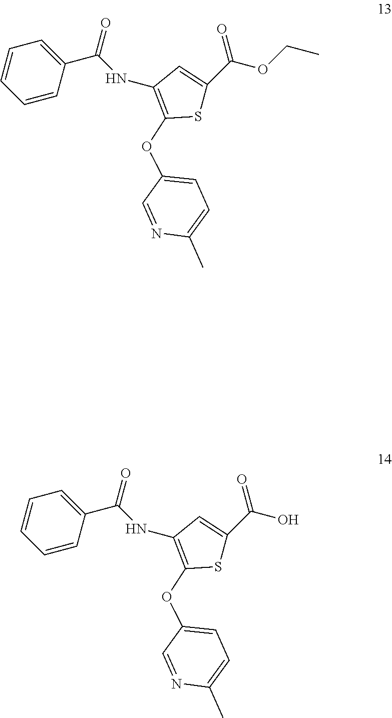 [SMILES: CCOC(=O)c1cc(NC(=O)c2ccccc2)c(Oc2ccc(C)nc2)s1 ; Cc1ccc(Oc2sc(C(=O)O)cc2NC(=O)c2ccccc2)cn1]

### Step 1: ethyl 5-((6-methylpyridin-3-yl)oxy)-4-(benzamido)thiophene-2-carboxylate (13)

Ethyl 5-((6-methylpyridin-3-yl)oxy)-4-nitrothiophene-2-carboxylate (0.15 g, 0.49 mmol) was dissolved in a mixed solvent of ethanol/water (10/1) (5 mL), NH₄Cl (0.26 g, 4.87 mmol) was added, and iron powder (0.27 g, 4.87 mmol) was slowly added at 0° C. After the addition was completed, the mixture was stirred for 30 min and heated at reflux at 85° C. for 4 h. After the reaction was completed, the mixture was filtered through celite and concentrated under reduced pressure to remove the solvent, thereby giving a yellow oil.

The product obtained in the previous step was dissolved in 4 mL of anhydrous tetrahydrofuran solvent, and triethylamine (0.98 g, 0.97 mmol) and benzoyl chloride (0.85 g, 0.63 mmol) were slowly added dropwise at 0° C. After the addition was completed, the mixture was stirred at room temperature for 4 h. After the reaction was completed, water was added, and the mixture was extracted with ethyl acetate (3 times). The organic phases were combined, washed with saturated brine, and dried over anhydrous Na₂SO₄. The resulting mixture was purified by silica gel column chromatography (petroleum ether/ethyl acetate=8:1) to give a pale yellow solid (0.11 g, 57.6% yield). ¹H NMR (300 MHz, DMSO-d₆) δ 10.36 (s, 1H), 8.55 (d, J=2.9 Hz, 1H), 7.95 (s, 1H), 7.83 (m, 3H), 7.63-7.44 (m, 4H), 4.29 (q, J=7.1 Hz, 2H), 2.53 (s, 3H), 1.28 (t, J=7.1 Hz, 3H).

### Step 2: 5-((6-methylpyridin-3-yl)oxy)-4-(benzamido)thiophene-2-carboxylic acid (14)

Ethyl 5-((6-methylpyridin-3-yl)oxy)-4-(benzamido)thiophene-2-carboxylate (100.00 mg, 0.262 mmol) was dissolved in a mixed solvent of methanol/tetrahydrofuran (1/1) (2 mL), and a 4 mol/L LiOH solution (1 mL) was added. After the addition was completed, the mixture was stirred at room temperature for 5 h. After the reaction was completed, 1 N HCl was added to adjust the pH of the solution to 4. The mixture was stirred for another 30 mz and filtered to give a white solid (83.5 mg, 90.1% yield). ¹H NMR (300 MHz, DMSO-d₆) δ 10.27 (s, 1H), 8.38 (d, J=3.1 Hz, 1H), 7.84 (dd, J=9.6, 2.6 Hz, 3H), 7.62-7.50 (m, 2H), 7.50-7.41 (i, 2H), 7.28 (d, J=8.6 Hz, 1H), 2.43 (s, 3H).

### Example 15

The following compounds were prepared similarly as in Example 13 and Example 14:

| No. | Structural formula | Nuclear magnetic resonance data |
|---|---|---|
| 15-1 | 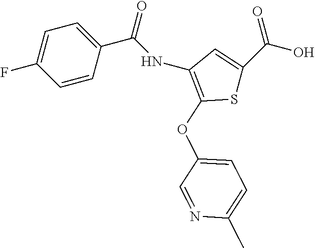 [SMILES: Cc1ccc(Oc2sc(C(=O)O)cc2NC(=O)c2ccc(F)cc2)cn1] | ¹H NMR (300 MHz, DMSO-d₆) δ 13.27 (s, 1H), 10.27 (s, 1H), 8.38 (d, J = 3.0 Hz, 1H), 7.93 (dd, J = 8.9, 5.4 Hz, 2H), 7.85 (s, 1H), 7.53 (dd, J = 8.5, 3.0 Hz, 1H), 7.33 (m, 3H), 2.45 (s, 3H). |
| 15-2 | 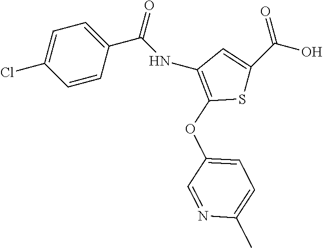 [SMILES: Cc1ccc(Oc2sc(C(=O)O)cc2NC(=O)c2ccc(Cl)cc2)cn1] | ¹H NMR (300 MHz, DMSO-d₆) δ 10.34 (s, 1H), 8.40 (d, J = 3.1 Hz, 1H), 7.85 (d, J = 1.6 Hz, 3H), 7.62-7.51 (m, 3H), 7.33 (d, J = 8.6 Hz, 1H), 2.46 (s, 3H). |
| 15-3 | 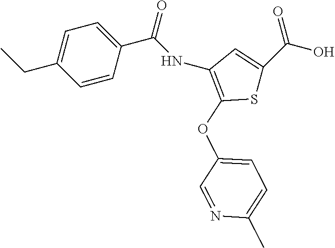 [SMILES: CCc1ccc(C(=O)Nc2cc(C(=O)O)sc2Oc2ccc(C)nc2)cc1] | ¹H NMR (300 MHz, DMSO-d₆) δ 10.15 (s, 1H), 8.39 (d, J = 3.0 Hz, 1H), 7.84 (s, 1H), 7.77 (d, J = 8.3 Hz, 2H), 7.54 (dd, J = 8.5, 2.9 Hz, 1H), 7.31 (d, J = 8.1 Hz, 3H), 2.65 (q, J = 7.5 Hz, 2H), 2.45 (s, 3H), 1.18 (t, J = 7.6 Hz, 3H). |
| 15-4 | 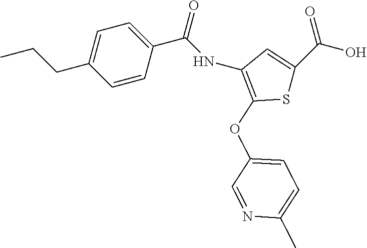 [SMILES: CCCc1ccc(C(=O)Nc2cc(C(=O)O)sc2Oc2ccc(C)nc2)cc1] | ¹H NMR (300 MHz, DMSO-d₆) δ 10.18 (s, 1H), 8.45 (d, J = 3.0 Hz, 1H), 7.84 (s, 1H), 7.76 (d, J = 8.3 Hz, 2H), 7.66 (dd, J = 8.6, 2.9 Hz, 1H), 7.41 (d, J = 8.7 Hz, 1H), 7.29 (d, J = 8.3 Hz, 2H), 2.60 (t, J = 7.5 Hz, 2H), 2.48 (s, 3H), 1.59 (h, J = 7.3 Hz, 2H), 0.88 (t, J = 7.3 Hz, 3H). |
| 15-5 | 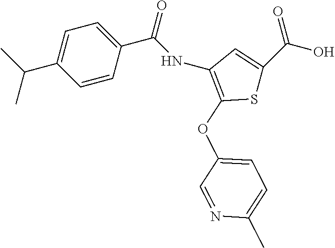 [SMILES: Cc1ccc(Oc2sc(C(=O)O)cc2NC(=O)c2ccc(C(C)C)cc2)cn1] | ¹H NMR (300 MHz, DMSO-d₆) δ 10.26 (s, 1H), 8.47 (d, J = 3.0 Hz, 1H), 7.86 (s, 1H), 7.77 (d, J = 8.3 Hz, 2H), 7.72 (dd, J = 8.6, 2.9 Hz, 1H), 7.45 (d, J = 8.8 Hz, 1H), 7.33 (d, J = 8.3 Hz, 2H), 2.92 (p, J = 7.0 Hz, 1H), 1.19 (d, J = 6.9 Hz, 6H). |
| 15-6 | 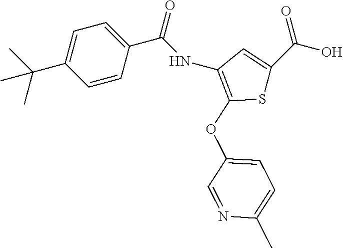 [SMILES: Cc1ccc(Oc2sc(C(=O)O)cc2NC(=O)c2ccc(C(C)(C)C)cc2)cn1] | ¹H NMR (300 MHz, DMSO-d₆) δ 10.23 (s, 1H), 8.48 (d, J = 3.0 Hz, 1H), 7.86 (s, 1H), 7.81-7.74 (m, 2H), 7.70 (dd, J = 8.6, 3.0 Hz, 1H), 7.50 (d, J = 8.5 Hz, 2H), 7.45 (d, J = 8.6 Hz, 1H), 2.50 (s, 3H), 1.29 (s, 9H). |
| 15-7 | 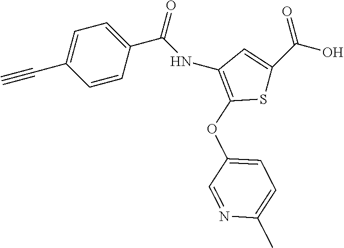 [SMILES: C#Cc1ccc(C(=O)Nc2cc(C(=O)O)sc2Oc2ccc(C)nc2)cc1] | ¹H NMR (300 MHz, DMSO-d₆) δ 10.34 (s, 1H), 8.38 (d, J = 3.1 Hz, 1H), 7.84 (t, J = 4.3 Hz, 3H), 7.63-7.49 (m, 3H), 7.31 (d, J = 8.6 Hz, 1H), 4.42 (s, 1H), 2.45 (s, 3H). |
| 15-8 | 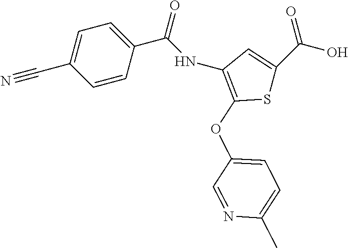 [SMILES: Cc1ccc(Oc2sc(C(=O)O)cc2NC(=O)c2ccc(C#N)cc2)cn1] | ¹H NMR (300 MHz, DMSO-d₆) δ 13.32 (s, 1H), 10.55 (s, 1H), 8.39 (d, J = 3.0 Hz, 1H), 7.99 (s, 4H), 7.88 (s, 1H), 7.54 (dd, J = 8.5, 3.0 Hz, 1H), 7.31 (d, J = 8.5 Hz, 1H), 2.45 (s, 3H). |
| 15-9 | 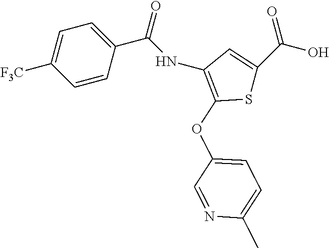 [SMILES: Cc1ccc(Oc2sc(C(=O)O)cc2NC(=O)c2ccc(C(F)(F)F)cc2)cn1] | ¹H NMR (300 MHz, DMSO-d₆) δ 10.49 (s, 1H), 8.38 (d, J = 3.0 Hz, 1H), 8.03 (d, J = 8.3 Hz, 2H), 7.87 (d, J = 7.0 Hz, 3H), 7.53 (dd, J = 8.5, 3.0 Hz, 1H), 7.29 (d, J = 8.5 Hz, 1H), 2.44 (s, 3H). |
| 15-10 | 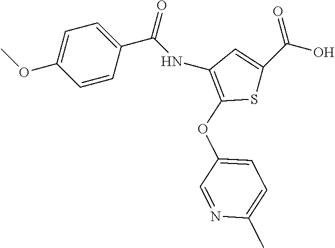 [SMILES: COc1ccc(C(=O)Nc2cc(C(=O)O)sc2Oc2ccc(C)nc2)cc1] | ¹H NMR (300 MHz, DMSO-d₆) δ 13.21 (s, 1H), 10.06 (s, 1H), 8.37 (d, J = 3.0 Hz, 1H), 7.84 (d, J = 8.6 Hz, 3H), 7.51 (dd, J = 8.6, 3.0 Hz, 1H), 7.29 (d, J = 8.7 Hz, 1H), 7.01 (d, J = 9.0 Hz, 2H), 3.81 (s, 3H), 2.44 (s, 3H). |
| 15-11 | 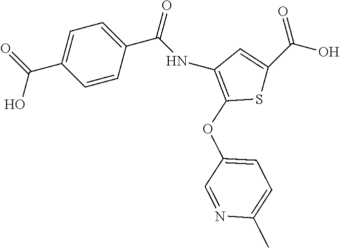 [SMILES: Cc1ccc(Oc2sc(C(=O)O)cc2NC(=O)c2ccc(C(=O)O)cc2)cn1] | ¹H NMR (300 MHz, DMSO-d₆) δ 10.45 (s, 1H), 8.38 (d, J = 3.1 Hz, 1H), 8.02 (d, J = 8.4 Hz, 2H), 7.93 (d, J = 8.4 Hz, 2H), 7.86 (s, 1H), 7.54 (dd, J = 8.6, 3.1 Hz, 1H), 7.30 (d, J = 8.6 Hz, 1H), 2.44 (s, 3H). |

### Example 13 and Example 14

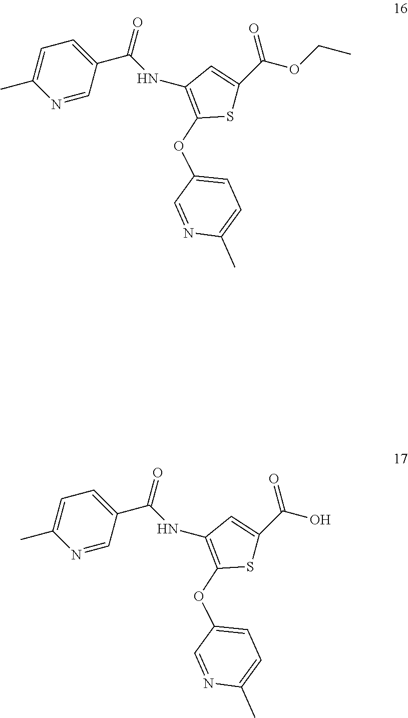

### Step 1: ethyl 4-(6-methylnicotinamido)-5-((6-methylpyridin-3-yl)oxy)thiophene-2-carboxylate (16)

Ethyl 5-((6-methylpyridin-3-yl)oxy)-4-nitrothiophene-2-carboxylate (0.15 g, 0.49 mmol) was dissolved in a mixed solvent of ethanol/water (10/1) (5 mL), NH₄Cl (0.26 g, 4.87 mmol) was added, and iron powder (0.27 g, 4.87 mmol) was slowly added at 0° C. After the addition was completed, the mixture was stirred for 30 min and heated at reflux at 85° C. for 4 h. After the reaction was completed, the mixture was filtered through celite and concentrated under reduced pressure to remove the solvent, thereby giving a yellow oil.

The product obtained in the previous step was dissolved in 4 mL of anhydrous dichloromethane solvent, and 6-methylnicotinamide (0.10 g, 0.73 mmol), triethylamine (0.10 g, 0.97 mmol), and Castro's condensation agent (PyBOP) (0.38 mg, 0.73 mmol) were sequentially added. After the addition was completed, the mixture was stirred at room temperature for 10 h. After the reaction was completed, the mixture was filtered to remove the insoluble substances. Water was added to the filtrate, and the mixture was extracted with ethyl acetate (3 times). The organic phases were combined, washed with saturated brine, and dried over anhydrous Na₂SO₄. The resulting mixture was purified by silica gel column chromatography (petroleum ether/ethyl acetate=2:1) to give a white solid (0.09 g, 43.6% yield). ¹H NMR (300 MHz, DMSO-d₆) δ 10.43 (s, 1H), 8.87 (d, J=3.0 Hz, 1H), 8.40 (d, J=2.9 Hz, 1H), 8.09 (dd, J=8.1, 2.4 Hz, 1H), 7.96 (s, 1H), 7.56 (dd, J=8.5, 3.0 Hz, 1H), 7.39 (d, J=8.1 Hz, 1H), 7.31 (d, J=8.5 Hz, 1H), 4.28 (q, J=7.1 Hz, 2H), 2.53 (s, 3H), 2.45 (s, 3H), 1.28 (t, J=7.1 Hz, 3H).

### Step 2: 4-(6-methylnicotinamido)-5-((6-methylpyridin-3-yl)oxy)thiophene-2-carboxylic acid (17)

Ethyl 4-(6-methylnicotinamido)-5-((6-methylpyridin-3-yl)oxy)thiophene-2-carboxylate (100.00 mg, 0.262 mmol) was dissolved in a mixed solvent of methanol/tetrahydrofuran (1/1) (2 mL), and a 4 mol/L LiOH solution (1 mL) was added. After the addition was completed, the mixture was stirred at room temperature for 5 h. After the reaction was completed, 1 N HCl was added to adjust the pH of the solution to 5-6. The mixture was stirred for another 30 min and filtered to give a white solid (56.0 mg, 60.3% yield). ¹H NMR (300 MHz, DMSO-d₆) δ 10.11 (s, 1H), 8.54 (s, 1H), 8.04 (d, J=3.0 Hz, 1H), 7.76 (d, J=9.6 Hz, 1H), 7.53 (s, 1H), 7.19 (dd, J=8.5, 3.1 Hz, 1H), 7.03 (d, J=8.1 Hz, 1H), 6.95 (d, J=8.5 Hz, 1H), 2.18 (s, 3H), 2.10 (s, 3H).

### Example 18

The following compounds were prepared similarly as in Example 16 and Example 17:

| No. | Structural formula | Nuclear magnetic resonance data |
|---|---|---|
| 18-1 | 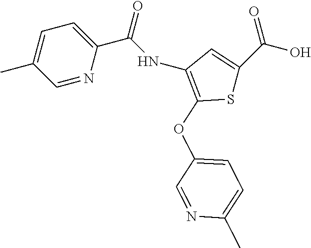 [SMILES: Cc1ccc(C(=O)Nc2cc(C(=O)O)sc2Oc2ccc(C)nc2)nc1] | ¹H NMR (300 MHz, DMSO-d₆) δ 10.32 (s, 1H), 8.51 (d, J = 2.2 Hz, 1H), 8.44 (d, J = 3.0 Hz, 1H), 8.01 (d, J = 7.0 Hz, 2H), 7.85 (dd, J = 8.0, 2.5 Hz, 1H), 7.61 (dd, J = 8.5, 3.0 Hz, 1H), 7.31 (d, J = 8.5 Hz, 1H), 2.46 (s, 3H), 2.39 (s, 3H). |
| 18-2 | 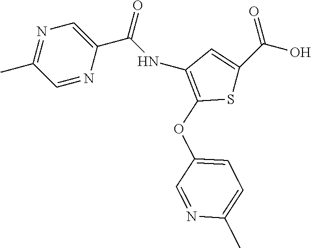 [SMILES: Cc1ccc(Oc2sc(C(=O)O)cc2NC(=O)c2cnc(C)cn2)cn1] | ¹H NMR (300 MHz, DMSO-d₆) δ 10.46 (s, 1H), 9.10 (d, J = 1.6 Hz, 1H), 8.65 (d, J = 1.6 Hz, 1H), 8.42 (d, J = 3.0 Hz, 1H), 7.92 (s, 1H), 7.60 (dd, J = 8.5, 3.0 Hz, 1H), 7.31 (d, J = 8.6 Hz, 1H), 2.60 (s, 3H), 2.45 (s, 3H). |
| 18-3 | 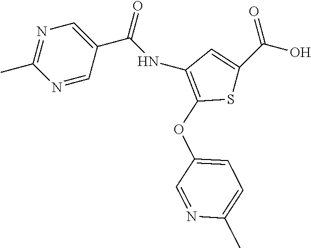 [SMILES: Cc1ccc(Oc2sc(C(=O)O)cc2NC(=O)c2cnc(C)nc2)cn1] | ¹H NMR (300 MHz, DMSO-d₆) δ 10.61 (s, 1H), 9.04 (s, 2H), 8.41 (d, J = 3.0 Hz, 1H), 7.91 (s, 1H), 7.58 (dd, J = 8.6, 3.0 Hz, 1H), 7.33 (d, J = 8.6 Hz, 1H), 2.68 (s, 3H), 2.45 (s, 3H). |
| 18-4 | 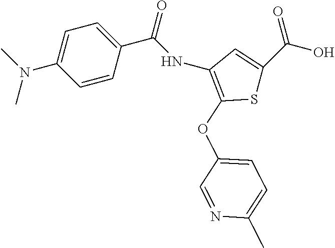 [SMILES: Cc1ccc(Oc2sc(C(=O)O)cc2NC(=O)c2ccc(N(C)C)cc2)cn1] | ¹H NMR (300 MHz, DMSO-d₆) δ 9.82 (s, 1H), 8.36 (d, J = 2.9 Hz, 1H), 7.83 (s, 1H), 7.74 (d, J = 9.0 Hz, 2H), 7.49 (dd, J = 8.5, 3.0 Hz, 1H), 7.28 (d, J = 8.5 Hz, 1H), 6.70 (d, J = 9.2 Hz, 2H), 2.97 (s, 6H), 2.43 (s, 3H). |
| 18-5 | 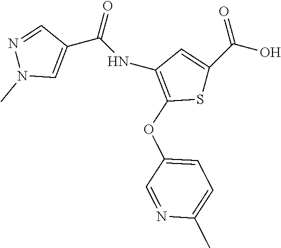 [SMILES: Cc1ccc(Oc2sc(C(=O)O)cc2NC(=O)c2cnn(C)c2)cn1] | ¹H NMR (300 MHz, DMSO-d₆) δ 13.28 (s, 1H), 9.88 (s, 1H), 8.37 (d, J = 3.0 Hz, 1H), 8.26 (s, 1H), 7.92 (s, 1H), 7.84 (s, 1H), 7.50 (dd, J = 8.5, 3.0 Hz, 1H), 7.29 (d, J = 8.6 Hz, 1H), 3.85 (s, 3H), 2.44 (s, 3H). |

The pharmacological experiments and results of some compounds of general formula I in the present disclosure are as follows:

### Procedures:

An HEK293 cell strain stably transfected with the P2Y₁₄ receptor was cultured in a DMEM culture medium (containing 1000 fetal bovine serum, 100 U/mL penicillin, and 100 μg/mL streptomycin). Before the experiment, the cells were seeded into a culture plate at a density of 1×10⁵ cells/well, and a serum-free culture medium was used instead. The cells were cultured at 37° C. with 9500 02 and 5% CO₂ humidity. IBMX was added to inhibit the activity of PDEs, thereby ensuring a relatively high level of cAMP. The AC agonist Forskolin (30 μM) was adopted to stimulate the production of cellular cAMP. Test compounds at different concentrations (0.01 nm, 0.1 nm, 1 nm, 10 nm, and 100 nm) were added in advance, and PPTN was used as a positive control. Subsequently, 1 μM P2Y₁₄ receptor agonist UDPG was added, and after 4 h, the content of intracellular cAMP was measured using a cAMP Glo™ Assay kit (PROMEGA Co. Ltd., USA). The inhibition rate was calculated based on the cAMP content.

TABLE 1
Inhibition rates of some compounds on
P2Y₁₄ receptor at the cellular level:

| No. | Inhibition @ 100 nM |
|---|---|
| 2 | 70.40% |
| 3-1 | 44.14% |
| 3-2 | 69.61% |
| 3-3 | 66.04% |
| 3-4 | 30.93% |
| 3-5 | 36.21% |
| 3-6 | 40.44% |
| 3-7 | 62.62% |
| 3-8 | 52.83% |
| 3-9 | 57.06% |
| 3-10 | 32.73% |
| 3-11 | 34.45% |
| 3-12 | 44.14% |
| 3-13 | 46.24% |
| 3-14 | 62.81% |
| 3-15 | 88.39% |
| 3-16 | 55.84% |
| 3-17 | 45.83% |
| 3-18 | 69.80% |
| 3-19 | 67.36% |
| 3-20 | 66.32% |
| 3-21 | 54.29% |
| 3-22 | 34.62% |
| 3-23 | 81.09% |
| 3-24 | 75.64% |
| 3-25 | 49.53% |
| 5 | 99.37% |
| 6-1 | 61.99% |
| 6-2 | 88.39% |
| 6-3 | 74.88% |
| 6-4 | 81.35% |
| 6-5 | 75.54% |
| 6-6 | 43.55% |
| 6-7 | 60.26% |
| 8 | 75.62% |
| 9-1 | 82.35% |
| 9-2 | 55.67% |
| 9-3 | 71.45% |
| 9-4 | 59.76% |
| 11 | 75.64% |
| 12-1 | 57.55% |
| 12-2 | 61.38% |
| 12-3 | 67.55% |
| 12-4 | 82.45% |
| 14 | 86.87% |
| 15-1 | 75.65% |
| 15-2 | 75.44% |
| 15-3 | 46.44% |
| 15-4 | 85.64% |
| 15-5 | 78.65% |
| 15-6 | 86.14% |
| 15-7 | 94.63% |
| 15-8 | 76.44% |
| 15-9 | 82.14% |
| 15-10 | 91.34% |
| 15-11 | 79.31% |
| 17 | 65.11% |
| 18-1 | 45.11% |
| 18-2 | 48.19% |
| 18-3 | 56.17% |
| 18-4 | 46.78% |
| 18-5 | 45.75% |

### Experimental Method for Pharmacological Study of Inhibition of LPS-Induced Macrophage Inflammatory Response by Test Compounds

Human THP-1 cells were cultured in an RPMI-1640 culture medium (containing 10% fetal bovine serum, 100 U/mL penicillin, and 100 μg/mL streptomycin) and seeded into a culture plate at a density of 1×10⁵ cells/well before the experiment. The cells were cultured at 37° C. with 95% 02 and 5% CO₂ humidity. Before the experiment, PMA was added at 100 ng/mL to each well, and the cells were incubated for 24 h to induce the differentiation of the THP-1 cells into macrophages. Test compound 5 (2.5 μM, 5 μM, and 10 μM) and PPTN (5 μM) were added to the culture medium in advance for intervention. After 1 h, LPS at a final concentration of 100 ng/mL was added to the cells. After 3 h, ATP at a final concentration of 5 mM was added. After 1 h, the indexes were determined as follows:

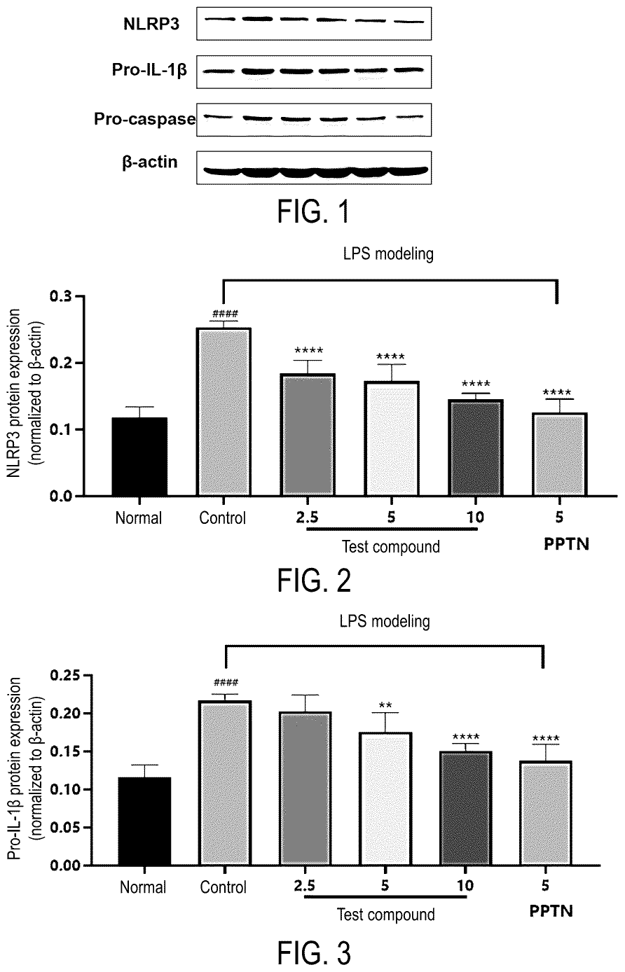

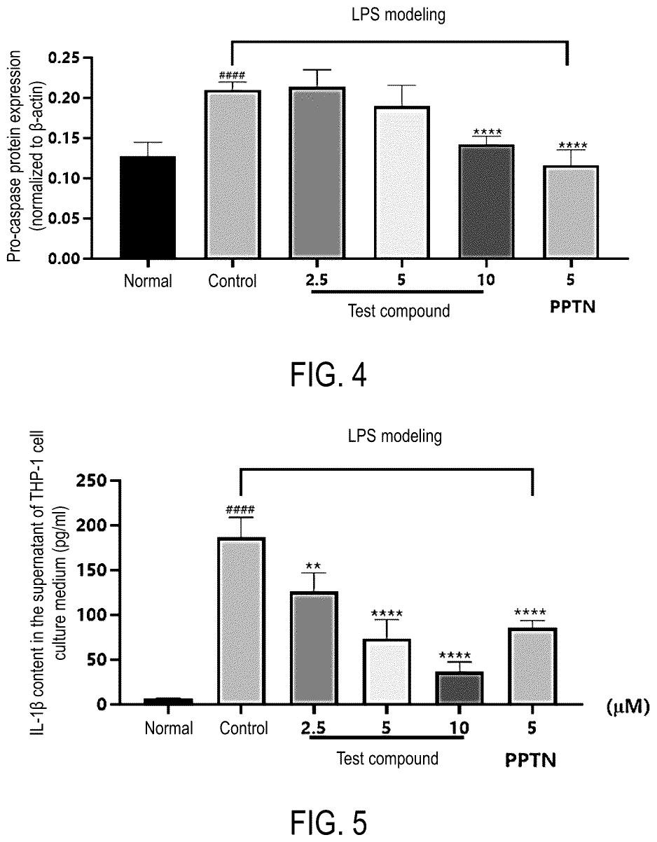

The expression of proteins downstream of the UDPG/P2Y₁₄ receptor signaling pathway in cells was detected by Western Blot, and the results are shown in FIGS. 1-4. LPS caused a significant increase in the expression of related proteins downstream of the P₂Y14 receptor in THP-1 cells, indicating that the modeling was successful; different doses of the test compounds were able to down-regulate the expression of related proteins downstream of the P₂Y14 receptor to different degrees, and showed significant differences compared with the model control group; PPTN also showed an expected effect, indicating that the experimental results were true and reliable.

The level of IL-1β in the supernatant of the cell culture medium was detected by an ELISA kit (Shenzhen NeoBioscience Technology Co., Ltd.), and the results are shown in FIG. 5. LPS caused a significant increase in the level of IL-1β in the supernatant of the THP-1 cell culture medium, indicating that the modeling was successful; different doses of the test compounds were able to down-regulate the level of IL-1β in the supernatant of the cell culture medium to different degrees, and showed significant differences compared with the model control group; PPTN also showed an expected effect, indicating that the experimental results were true and reliable.

### Method for Pharmacological Experimental Study of Therapeutic Effects of Test Compounds on Acute Peritonitis at the Whole Animal Level

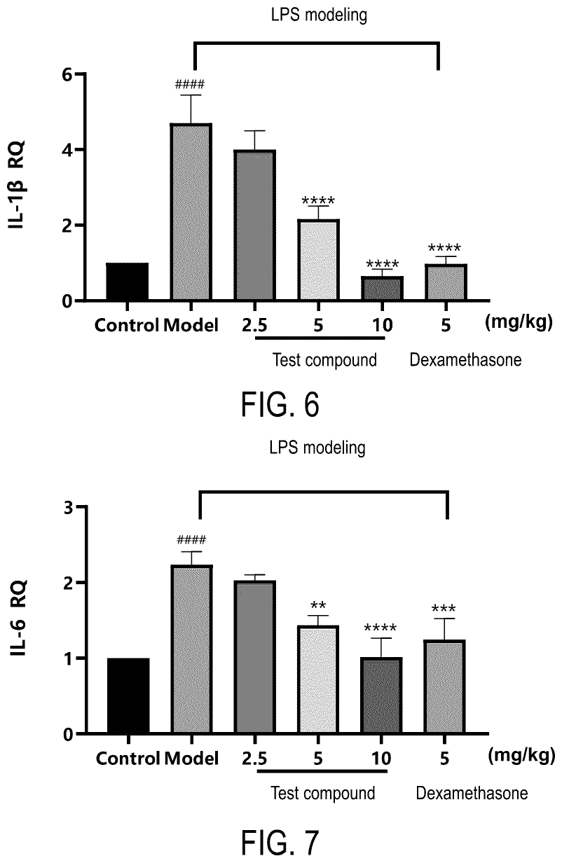

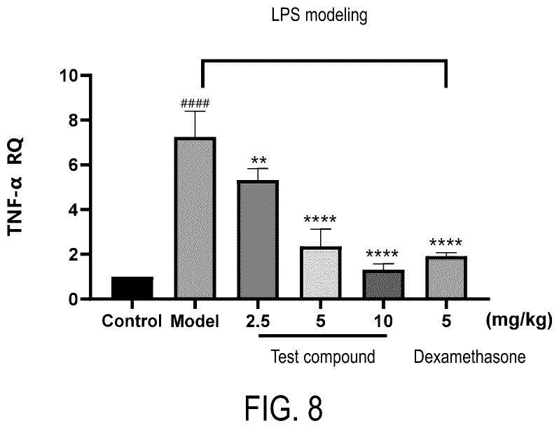

Male clean-grade ICR mice were housed with free access to water and food, and with illumination for 12 h every day at an ambient temperature of 25±2° C. The animals were divided into several groups, that is, a normal control group, a model control group, and administration groups (test compound and dexamethasone). An acute peritonitis model was induced by a single intraperitoneal injection of LSP, while an equal volume of physiological saline was injected into the joint cavity in the normal control group. Each administration group was given test compound 5 (5 mg/kg, 10 mg/kg, and 20 mg/kg) and dexamethasone (10 mg/kg) by intraarticular injection. 6 h after the injection, 8 mL of PBS was injected, and the peritoneal cavity fluids were collected. The levels of inflammatory factors in the collected fluids were detected, and the results are shown in FIGS. 6-8. LPS caused a significant increase in the gene expression levels of inflammatory factors in mouse peritoneal fluids, thereby improving the success of modeling; different doses of the test compounds were able to down-regulate the gene expression levels of inflammatory factors to different degrees, and showed significant differences compared with the model control group; dexamethasone also showed an expected effect, indicating that the experimental results were true and reliable.

1. A thiophene-2-carboxylic acid derivative of general formula (I), and a tautomer thereof and a pharmaceutically acceptable salt thereof: 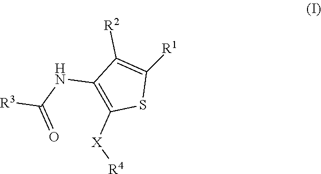
wherein R¹ is selected from COOR⁵;
R² is selected from H and halogen;
R³ is selected from phenyl, pyridinyl, pyrazinyl, pyrimidinyl, and imidazolyl, wherein the phenyl, pyridinyl, pyrazinyl, pyrimidinyl, or imidazolyl is independently optionally substituted with 1 to 5 substituents selected from: C₁₋₆ alkyl, halogen, cyano, carboxyl, acetyl, methanesulfonyl, C₁₋₄ haloalkyl, C₁₋₄ haloalkoxy, OH, C₁₋₆ alkoxy, and dimethylamino;
X is selected from O, S, and NH;
R⁴ is selected from: 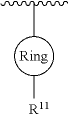
wherein Ring is phenyl, pyridinyl, pyridazinyl, pyrimidinyl, pyrazinyl, pyrrolyl, thienyl, furanyl, thiazolyl, isothiazolyl, imidazolyl, or pyrazolyl;
R⁵ is H, C₂₋₄ alkyl, C₂₋₃ alkyl-C₃₋₆ cycloalkyl, C₂₋₃OOCCH₃, C₂₋₃OCNMe₂, or C₂₋₃ NMe₂;
R⁷ is H;
R⁸ is selected from H, C₁₋₆ alkyl, C₃₋₆ cycloalkyl, CH₂CONH₂, CH₂COMe₂, CH₂CH₂OH, CH₂CH₂Me, (CH₂)₃OH, (CH₃)₃OMe, (CH₂)₄OH, (CH₂)₄OMe, (CH₂)₅OH, (CH₂)₂COOH, (CH₂)₃COOH, (CH₂)₄COOH, (CH₂)₅COOH, (CH₂)₂CH(CH₃)COOH, C(CH₂OH)₃, C(CH₂OH)₂CH₃, CH₂CH(CH₃)OH, (CH₂)₂CF₃, (CH₂)₃CF₃, (CH₂)₄CF₃, 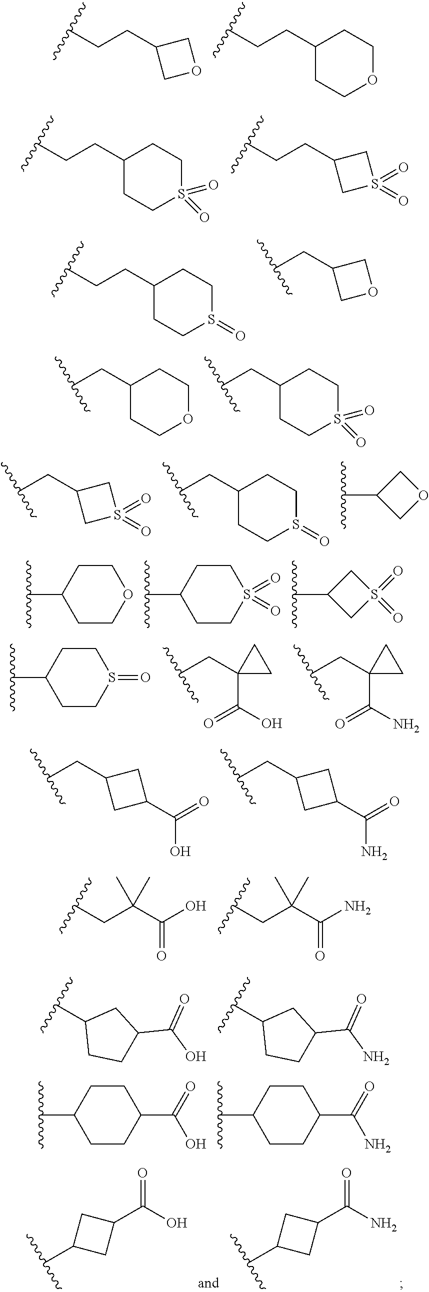
R¹¹ is selected from H, NO₂, CN, OH, NH₂, F, Cl, Br, I, C₁₋₆ alkyl, C₁₋₆ haloalkyl, C₁₋₆ alkoxy, C₁₋₆ haloalkoxy, COR⁸, CONR⁷R⁸, CO₂R⁸, SO₂NR⁷R⁸, and SO₃R⁸.

2. The thiophene-2-carboxylic acid derivative, and the tautomer thereof and the pharmaceutically acceptable salt thereof according to claim 1, wherein
R² is selected from H and F;
R⁵ is H, C₂H₅, C₂₋₃ alkyl-C₃₋₆ cycloalkyl, C₂₋₃OOCCH₃, C₂₋₃OCNMe₂, or C₂₋₃NMe₂.

3. The thiophene-2-carboxylic acid derivative, and the tautomer thereof and the pharmaceutically acceptable salt thereof according to claim 1, wherein
R³ is selected from phenyl, pyridinyl, pyrazinyl, pyrimidinyl, and imidazolyl, wherein the phenyl, pyridinyl, pyrazinyl, pyrimidinyl, or imidazolyl is independently optionally substituted with 1 to 2 substituents selected from: C₁₋₆ alkyl, halogen, cyano, C₁₋₄ haloalkyl, OH, C₁₋₆ alkoxy, and dimethylamino.

4. The thiophene-2-carboxylic acid derivative, and the tautomer thereof and the pharmaceutically acceptable salt thereof according to claim 3, wherein R⁴ is selected from: 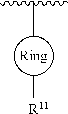
wherein Ring is phenyl, pyrimidinyl, pyridazinyl, or isothiazolyl;
R¹¹ is selected from H, NO₂, CN, OH, NH₂, F, Cl, Br, I, C₁₋₆ alkyl, C₁₋₆ haloalkyl, C₁₋₆ alkoxy, C₁₋₆ haloalkoxy, CONR⁷R⁸, CO₂R⁸, SO₂NR⁷R⁸, and SO₃R⁸;
R⁷ is H;
R⁸ is selected from H, C₁₋₆ alkyl, C₃₋₆ cycloalkyl, CH₂CONH₂, CH₂COMe₂, CH₂CH₂OH, CH₂CH₂Me, (CH₂)₃OH, (CH₃)₃OMe, (CH₂)₄OH, (CH₂)₄OMe, (CH₂)₂COOH, (CH₂)₃COOH, (CH₂)₄COOH, (CH₂)₂CH(CH₃)COOH, C(CH₂OH)₃, C(CH₂OH)₂CH₃, CH₂CH(CH₃)OH,

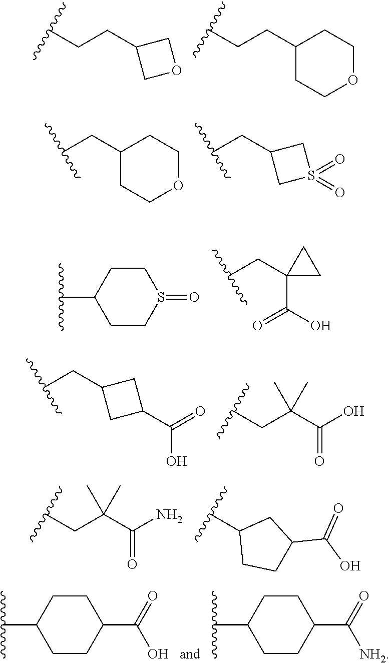

5. The thiophene-2-carboxylic acid derivative, and the tautomer thereof and the pharmaceutically acceptable salt thereof according to claim 1, wherein the compound of general formula (I) is selected from the following compounds:

| No. | Structural formula |
|---|---|
| 1 | 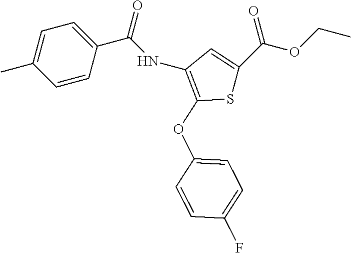 [SMILES: CCOC(=O)c1cc(NC(=O)c2ccc(C)cc2)c(Oc2ccc(F)cc2)s1] |
| 2 | 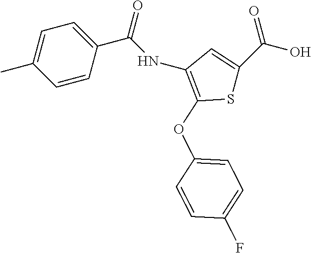 [SMILES: Cc1ccc(C(=O)Nc2cc(C(=O)O)sc2Oc2ccc(F)cc2)cc1] |
| 3-1 | 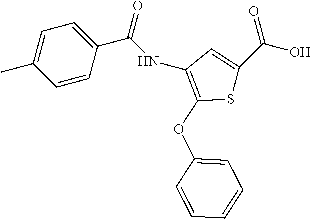 [SMILES: Cc1ccc(C(=O)Nc2cc(C(=O)O)sc2Oc2ccccc2)cc1] |
| 3-2 | 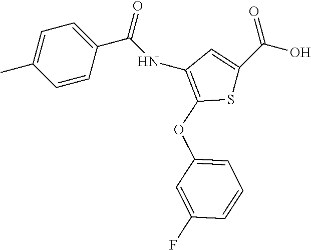 [SMILES: Cc1ccc(C(=O)Nc2cc(C(=O)O)sc2Oc2cccc(F)c2)cc1] |
| 3-3 | 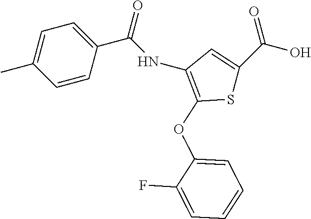 [SMILES: Cc1ccc(C(=O)Nc2cc(C(=O)O)sc2Oc2ccccc2F)cc1] |
| 3-4 | 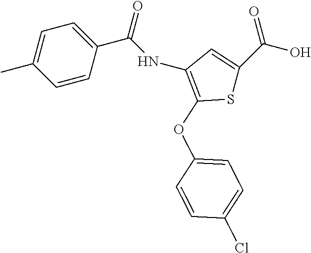 [SMILES: Cc1ccc(C(=O)Nc2cc(C(=O)O)sc2Oc2ccc(Cl)cc2)cc1] |
| 3-5 | 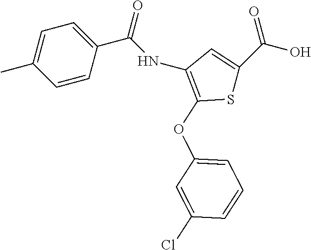 [SMILES: Cc1ccc(C(=O)Nc2cc(C(=O)O)sc2Oc2cccc(Cl)c2)cc1] |
| 3-6 | 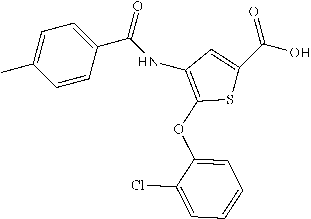 [SMILES: Cc1ccc(C(=O)Nc2cc(C(=O)O)sc2Oc2ccccc2Cl)cc1] |
| 3-7 | 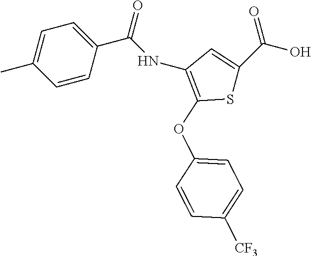 [SMILES: Cc1ccc(C(=O)Nc2cc(C(=O)O)sc2Oc2ccc(C(F)(F)F)cc2)cc1] |
| 3-8 | 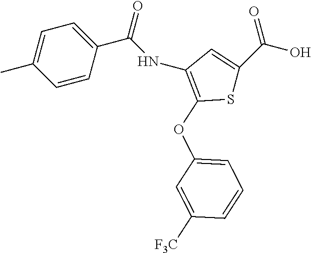 [SMILES: Cc1ccc(C(=O)Nc2cc(C(=O)O)sc2Oc2cccc(C(F)(F)F)c2)cc1] |
| 3-9 | 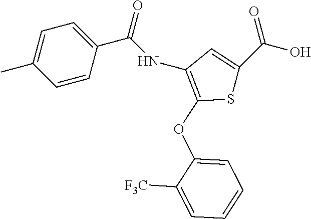 [SMILES: Cc1ccc(C(=O)Nc2cc(C(=O)O)sc2Oc2ccccc2C(F)(F)F)cc1] |
| 3-10 | 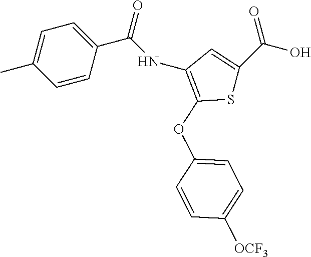 [SMILES: Cc1ccc(C(=O)Nc2cc(C(=O)O)sc2Oc2ccc(OC(F)(F)F)cc2)cc1] |
| 3-11 | 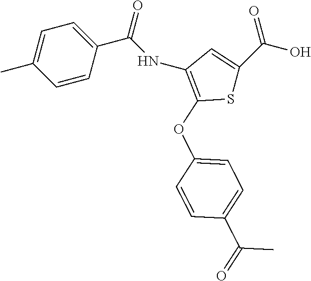 [SMILES: CC(=O)c1ccc(Oc2sc(C(=O)O)cc2NC(=O)c2ccc(C)cc2)cc1] |
| 3-12 | 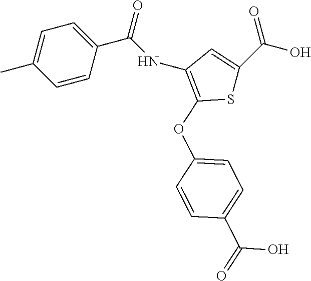 [SMILES: Cc1ccc(C(=O)Nc2cc(C(=O)O)sc2Oc2ccc(C(=O)O)cc2)cc1] |
| 3-13 | 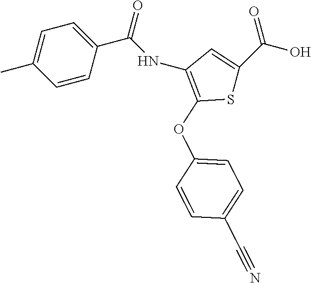 [SMILES: Cc1ccc(C(=O)Nc2cc(C(=O)O)sc2Oc2ccc(C#N)cc2)cc1] |
| 3-14 | 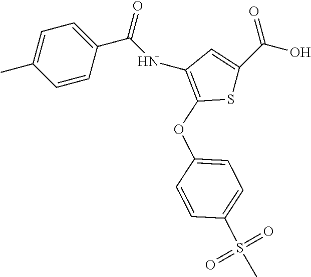 [SMILES: Cc1ccc(C(=O)Nc2cc(C(=O)O)sc2Oc2ccc(S(C)(=O)=O)cc2)cc1] |
| 3-15 | 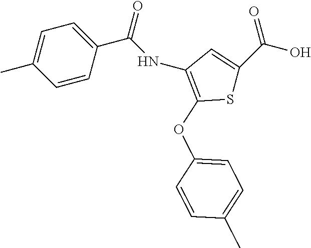 [SMILES: Cc1ccc(Oc2sc(C(=O)O)cc2NC(=O)c2ccc(C)cc2)cc1] |
| 3-16 | 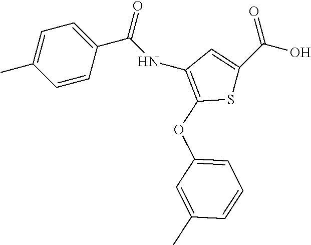 [SMILES: Cc1ccc(C(=O)Nc2cc(C(=O)O)sc2Oc2cccc(C)c2)cc1] |
| 3-17 | 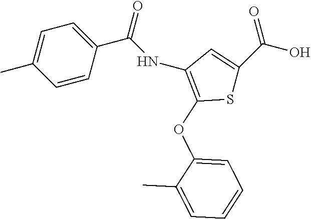 [SMILES: Cc1ccc(C(=O)Nc2cc(C(=O)O)sc2Oc2ccccc2C)cc1] |
| 3-18 | 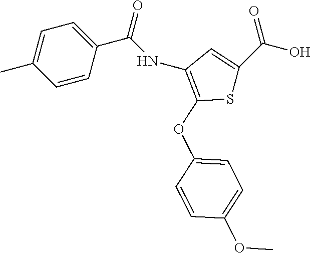 [SMILES: COc1ccc(Oc2sc(C(=O)O)cc2NC(=O)c2ccc(C)cc2)cc1] |
| 3-19 | 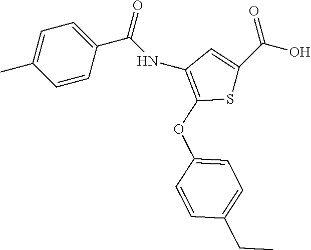 [SMILES: CCc1ccc(Oc2sc(C(=O)O)cc2NC(=O)c2ccc(C)cc2)cc1] |
| 3-20 | 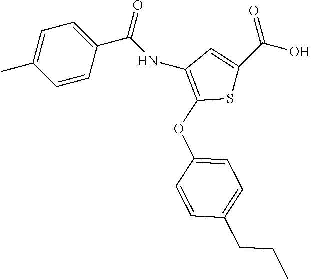 [SMILES: CCCc1ccc(Oc2sc(C(=O)O)cc2NC(=O)c2ccc(C)cc2)cc1] |
| 3-21 | 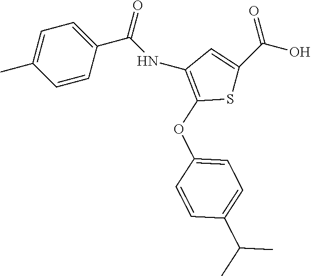 [SMILES: Cc1ccc(C(=O)Nc2cc(C(=O)O)sc2Oc2ccc(C(C)C)cc2)cc1] |
| 3-22 | 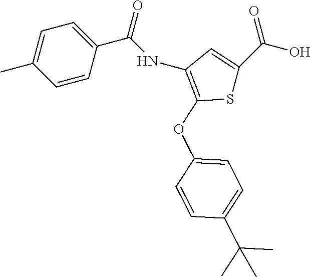 [SMILES: Cc1ccc(C(=O)Nc2cc(C(=O)O)sc2Oc2ccc(C(C)(C)C)cc2)cc1] |
| 3-23 | 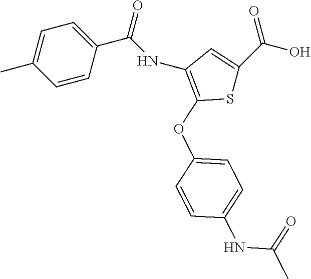 [SMILES: CC(=O)Nc1ccc(Oc2sc(C(=O)O)cc2NC(=O)c2ccc(C)cc2)cc1] |
| 3-24 | 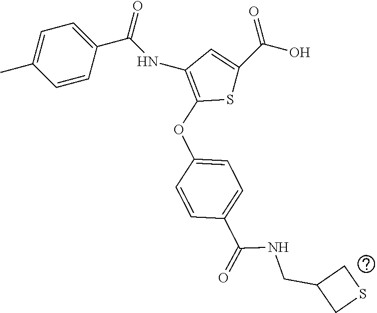 [SMILES: Cc1ccc(C(=O)Nc2cc(C(=O)O)sc2Oc2ccc(C(=O)NCC3CSC3)cc2)cc1] |
| 3-25 | 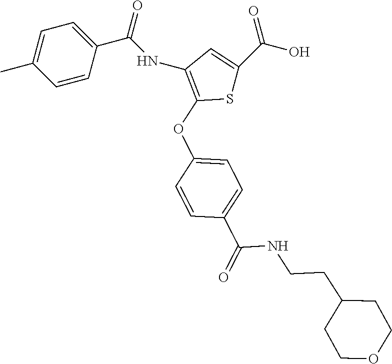 [SMILES: Cc1ccc(C(=O)Nc2cc(C(=O)O)sc2Oc2ccc(C(=O)NCCC3CCOCC3)cc2)cc1] |
| 4 | 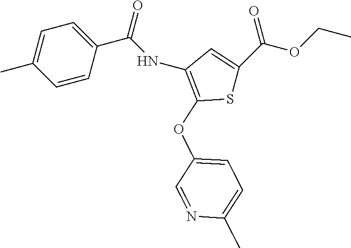 [SMILES: CCOC(=O)c1cc(NC(=O)c2ccc(C)cc2)c(Oc2ccc(C)nc2)s1] |
| 5 | 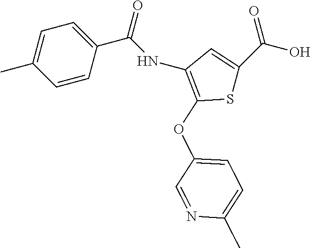 [SMILES: Cc1ccc(C(=O)Nc2cc(C(=O)O)sc2Oc2ccc(C)nc2)cc1] |
| 6-1 | 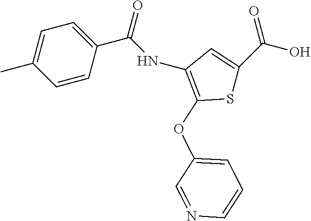 [SMILES: Cc1ccc(C(=O)Nc2cc(C(=O)O)sc2Oc2cccnc2)cc1] |
| 6-2 | 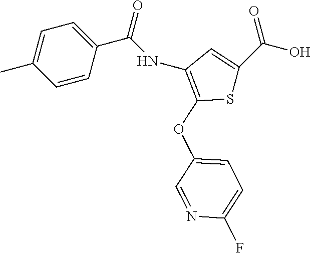 [SMILES: Cc1ccc(C(=O)Nc2cc(C(=O)O)sc2Oc2ccc(F)nc2)cc1] |
| 6-3 | 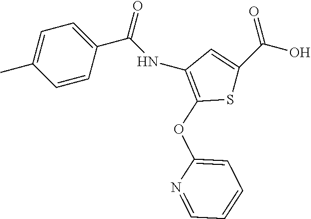 [SMILES: Cc1ccc(C(=O)Nc2cc(C(=O)O)sc2Oc2ccccn2)cc1] |
| 6-4 | 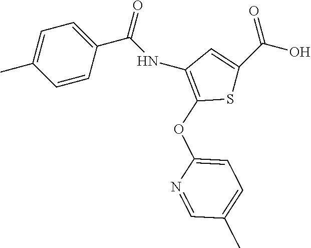 [SMILES: Cc1ccc(C(=O)Nc2cc(C(=O)O)sc2Oc2ccc(C)cn2)cc1] |
| 6-5 | 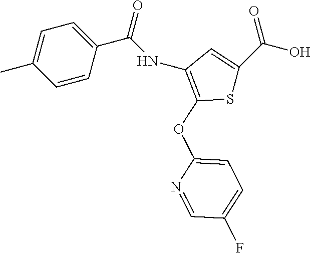 [SMILES: Cc1ccc(C(=O)Nc2cc(C(=O)O)sc2Oc2ccc(F)cn2)cc1] |
| 6-6 | 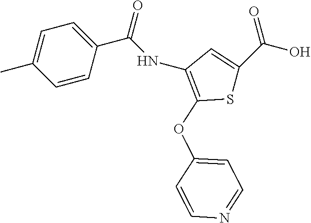 [SMILES: Cc1ccc(C(=O)Nc2cc(C(=O)O)sc2Oc2ccncc2)cc1] |
| 6-7 | 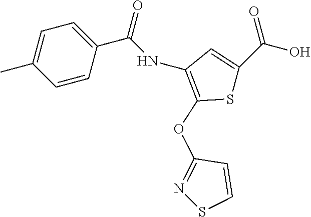 [SMILES: Cc1ccc(C(=O)Nc2cc(C(=O)O)sc2Oc2ccsn2)cc1] |
| 7 | 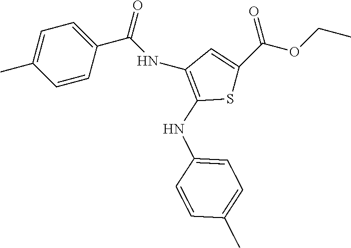 [SMILES: CCOC(=O)c1cc(NC(=O)c2ccc(C)cc2)c(Nc2ccc(C)cc2)s1] |
| 8 | 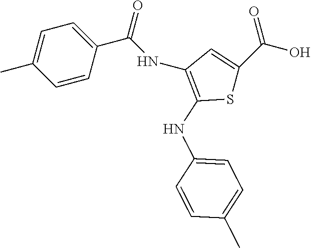 [SMILES: Cc1ccc(Nc2sc(C(=O)O)cc2NC(=O)c2ccc(C)cc2)cc1] |
| 9-1 | 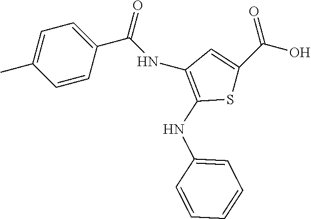 [SMILES: Cc1ccc(C(=O)Nc2cc(C(=O)O)sc2Nc2ccccc2)cc1] |
| 9-2 | 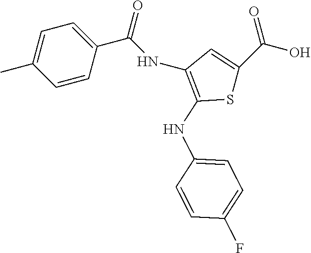 [SMILES: Cc1ccc(C(=O)Nc2cc(C(=O)O)sc2Nc2ccc(F)cc2)cc1] |
| 9-3 | 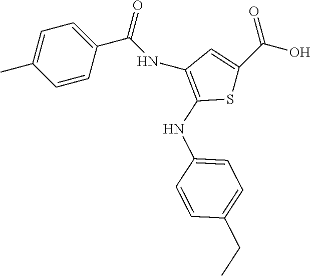 [SMILES: CCc1ccc(Nc2sc(C(=O)O)cc2NC(=O)c2ccc(C)cc2)cc1] |
| 9-4 | 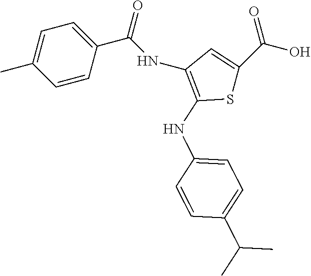 [SMILES: Cc1ccc(C(=O)Nc2cc(C(=O)O)sc2Nc2ccc(C(C)C)cc2)cc1] |
| 10 | 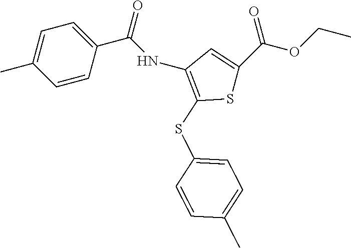 [SMILES: CCOC(=O)c1cc(NC(=O)c2ccc(C)cc2)c(Sc2ccc(C)cc2)s1] |
| 11 | 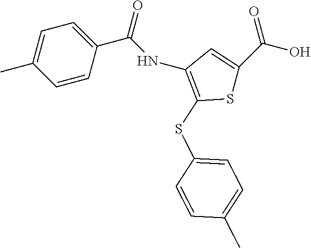 [SMILES: Cc1ccc(Sc2sc(C(=O)O)cc2NC(=O)c2ccc(C)cc2)cc1] |
| 12-1 | 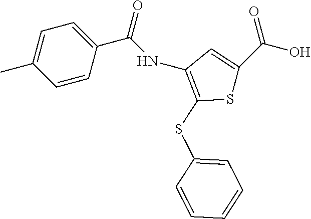 [SMILES: Cc1ccc(C(=O)Nc2cc(C(=O)O)sc2Sc2ccccc2)cc1] |
| 12-2 | 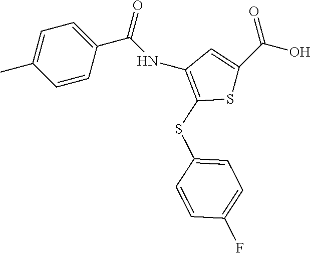 [SMILES: Cc1ccc(C(=O)Nc2cc(C(=O)O)sc2Sc2ccc(F)cc2)cc1] |
| 12-3 | 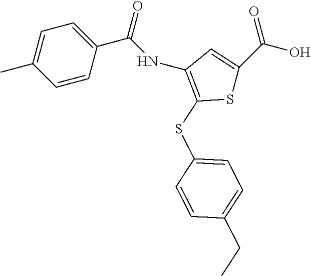 [SMILES: CCc1ccc(Sc2sc(C(=O)O)cc2NC(=O)c2ccc(C)cc2)cc1] |
| 12-4 | 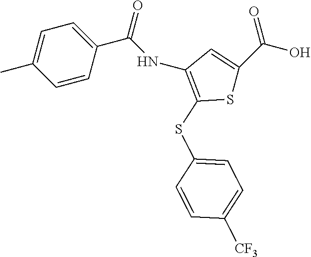 [SMILES: Cc1ccc(C(=O)Nc2cc(C(=O)O)sc2Sc2ccc(C(F)(F)F)cc2)cc1] |
| 13 | 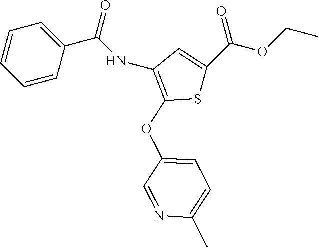 [SMILES: CCOC(=O)c1cc(NC(=O)c2ccccc2)c(Oc2ccc(C)nc2)s1] |
| 14 | 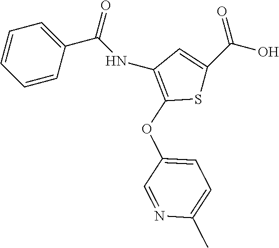 [SMILES: Cc1ccc(Oc2sc(C(=O)O)cc2NC(=O)c2ccccc2)cn1] |
| 15-1 | 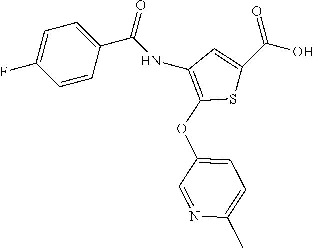 [SMILES: Cc1ccc(Oc2sc(C(=O)O)cc2NC(=O)c2ccc(F)cc2)cn1] |
| 15-2 | 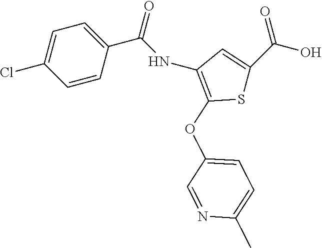 [SMILES: Cc1ccc(Oc2sc(C(=O)O)cc2NC(=O)c2ccc(Cl)cc2)cn1] |
| 15-3 | 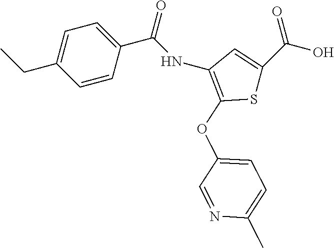 [SMILES: CCc1ccc(C(=O)Nc2cc(C(=O)O)sc2Oc2ccc(C)nc2)cc1] |
| 15-4 | 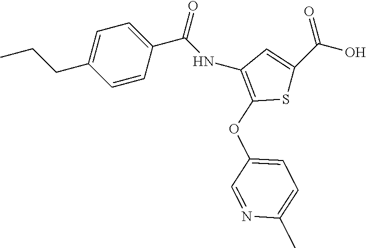 [SMILES: CCCc1ccc(C(=O)Nc2cc(C(=O)O)sc2Oc2ccc(C)nc2)cc1] |
| 15-5 | 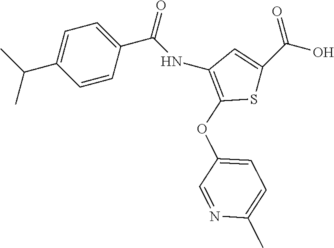 [SMILES: Cc1ccc(Oc2sc(C(=O)O)cc2NC(=O)c2ccc(C(C)C)cc2)cn1] |
| 15-6 | 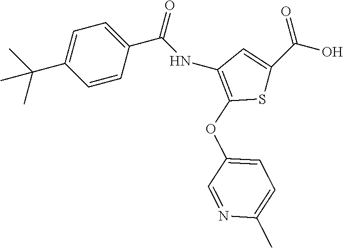 [SMILES: Cc1ccc(Oc2sc(C(=O)O)cc2NC(=O)c2ccc(C(C)(C)C)cc2)cn1] |
| 15-7 | 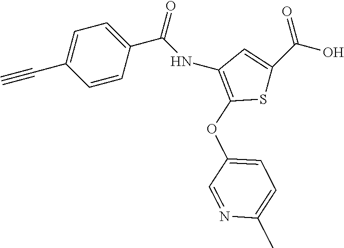 [SMILES: C#Cc1ccc(C(=O)Nc2cc(C(=O)O)sc2Oc2ccc(C)nc2)cc1] |
| 15-8 | 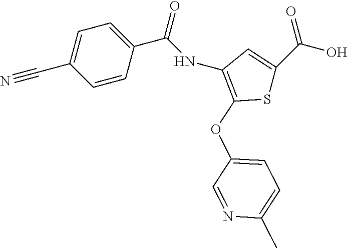 [SMILES: Cc1ccc(Oc2sc(C(=O)O)cc2NC(=O)c2ccc(C#N)cc2)cn1] |
| 15-9 | 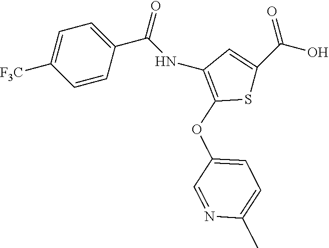 [SMILES: Cc1ccc(Oc2sc(C(=O)O)cc2NC(=O)c2ccc(C(F)(F)F)cc2)cn1] |
| 15-10 | 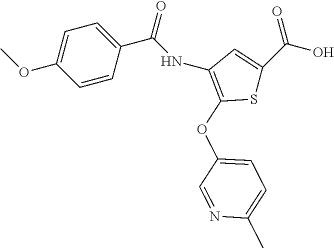 [SMILES: COc1ccc(C(=O)Nc2cc(C(=O)O)sc2Oc2ccc(C)nc2)cc1] |
| 15-11 | 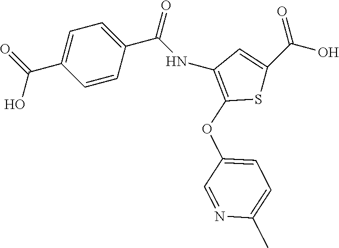 [SMILES: Cc1ccc(Oc2sc(C(=O)O)cc2NC(=O)c2ccc(C(=O)O)cc2)cn1] |
| 16 | 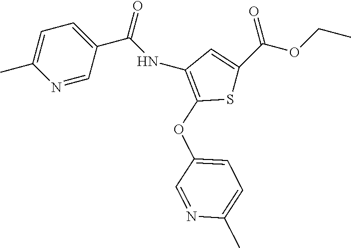 [SMILES: CCOC(=O)c1cc(NC(=O)c2ccc(C)nc2)c(Oc2ccc(C)nc2)s1] |
| 17 | 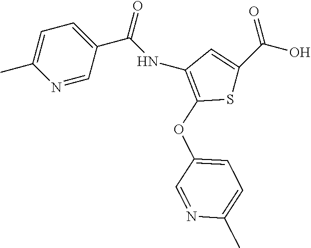 [SMILES: Cc1ccc(Oc2sc(C(=O)O)cc2NC(=O)c2ccc(C)nc2)cn1] |
| 18-1 | 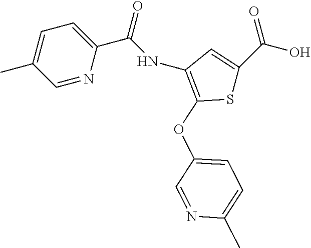 [SMILES: Cc1ccc(C(=O)Nc2cc(C(=O)O)sc2Oc2ccc(C)nc2)nc1] |
| 18-2 | 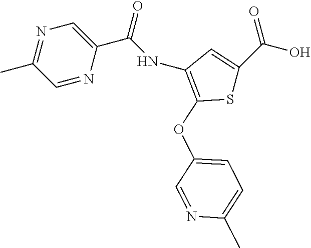 [SMILES: Cc1ccc(Oc2sc(C(=O)O)cc2NC(=O)c2cnc(C)cn2)cn1] |
| 18-3 | 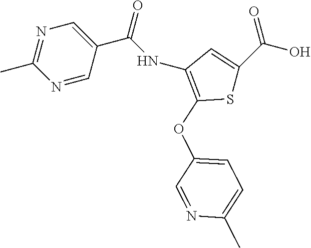 [SMILES: Cc1ccc(Oc2sc(C(=O)O)cc2NC(=O)c2cnc(C)nc2)cn1] |
| 18-4 | 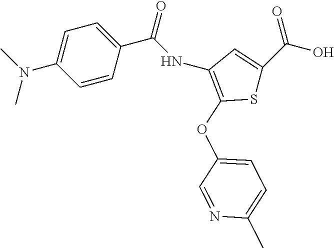 [SMILES: Cc1ccc(Oc2sc(C(=O)O)cc2NC(=O)c2ccc(N(C)C)cc2)cn1] |
| 18-5 | 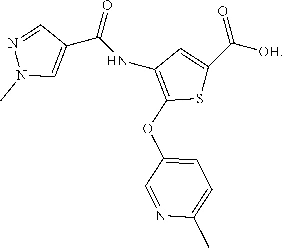 [SMILES: Cc1ccc(Oc2sc(C(=O)O)cc2NC(=O)c2cnn(C)c2)cn1] |

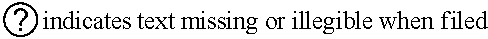

6. A preparation method for the thiophene-2-carboxylic acid derivative, and the tautomer thereof and the pharmaceutically acceptable salt thereof according to claim 1, comprising the following steps:
(1) subjecting a compound of general formula A to a nitration reaction to prepare a compound of general formula B;
(2) subjecting the compound of general formula B to a substitution reaction with different phenolic derivatives to prepare a compound of general formula C;
(3) subjecting the compound of general formula C to nitro reduction, then to a condensation reaction with different carboxylic acid or sulfonic acid derivatives or to a substitution reaction with different hydrocarbyl derivatives, and finally to a deprotection reaction to prepare a compound of general formula D; 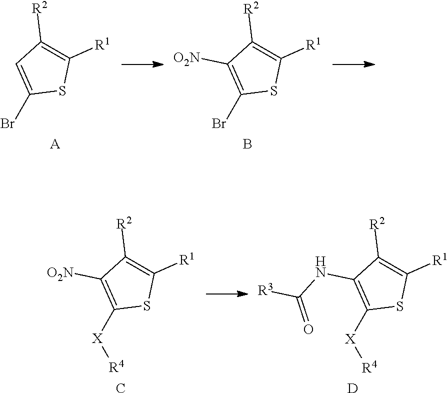
compound D is the compound of general formula (I), and R¹, R², R³, R⁴, and X are as defined in claim 1.

7. A pharmaceutical composition comprising the compound of general formula (I) or the pharmaceutically acceptable salt thereof according to claim 1, and a pharmaceutically acceptable excipient.

8. Use of the compound of general formula (I) or the pharmaceutically acceptable salt thereof according to claim 1 in the preparation of a P2Y₁₄ receptor antagonist drug.

9. Use of the compound of general formula (I) or the pharmaceutically acceptable salt thereof according to claim 1 in the preparation of a drug for treating an inflammatory disease.

10. The use according to claim 8, wherein the drug is formulated into different dosage forms by adding pharmaceutically acceptable excipients.

11. The use according to claim 9, wherein the drug is formulated into different dosage forms by adding pharmaceutically acceptable excipients.

12. Use of the pharmaceutical composition according to claim 7 in the preparation of a P2Y₁₄ receptor antagonist drug.

13. Use of the pharmaceutical composition according to claim 7 in the preparation of a drug for treating an inflammatory disease.

14. The use according to claim 12, wherein the drug is formulated into different dosage forms by adding pharmaceutically acceptable excipients.

15. The use according to claim 13, wherein the drug is formulated into different dosage forms by adding pharmaceutically acceptable excipients.
